(19) United States
(12) Patent Application Publication
(10) Pub. No.: US 2022/0080027 A1
(43) Pub. Date: Mar. 17, 2022

(54) METHODS OF TREATING NEURODEGENERATIVE DISEASES BY TARGETING THE PURINERGIC AND/OR ADENOSINE RECEPTORS

(71) Applicants: Board of Regents, The University of Texas System, Austin, TX (US); University of North Dakota, Grand Forks, ND (US)

(72) Inventors: Eliseo Eugenin, League City, TX (US); Jonathan Geiger, Grand Forks, ND (US); Stephani Velasquez, Clark, NJ (US)

(21) Appl. No.: 17/476,910

(22) Filed: Sep. 16, 2021

Related U.S. Application Data
(60) Provisional application No. 63/079,863, filed on Sep. 17, 2020.

(51) Int. Cl. A61K 38/17; A61K 38/46; A61P 31/18; A61P 25/28; C12N 15/86; G01N 33/50

(52) U.S. Cl. CPC A61K 38/1709; A61K 38/46; A61P 31/18; A61K 48/00; C12N 15/86; G01N 33/5038; A61P 25/28

(57) ABSTRACT

The present disclosure provides methods to reduce and/or increase the function of the purinergic/Adenosine system in individuals with detected cognitive impairment such as Alzheimer's, Parkinson's, and HIV as well as other diseases with dysregulated ATP secretion and its degradation products including adenosine. The present disclosure provides methods to block the toxic effects of increased circulating levels of ATP observed in several kinds of CNS diseases and maybe peripherical diseases.

### CROSS-REFERENCE TO RELATED APPLICATIONS

This application claims priority to U.S. Provisional Application Ser. No. 63/079,863, filed Sep. 17, 2020, the entire contents of which are incorporated herein by reference.

### STATEMENT REGARDING FEDERALLY SPONSORED RESEARCH

This invention was made with government support under MH096625 and NS105584 awarded by the National Institutes of Health. The government has certain rights in the invention.

### TECHNICAL FIELD OF THE INVENTION

The present invention relates in general to the field of treatment of cognitive impairment. More specifically, the present invention relates to preventing or reducing ATP concentrations in the circulation.

### BACKGROUND

Dementia is a significant public health concern worldwide. Currently, a combination of behavior, imaging, genetic testing, histology, and associated central nervous system (CNS) damage is used to diagnose different CNS diseases, including but not restricted to Alzheimer's, Parkinson's, ALS, Multiple Sclerosis, trauma, and NeuroHIV. Currently, biomarkery such as neurofilament light chain (NfL) and others can provide late evaluation of brain damage. However, most of these are unable to detect early onsets of brain damage, and several only detect late events as a confirmatory test.

What is needed are novel methods of treatment, methods of detection, an agents that can be used to treat dementia.

### SUMMARY OF THE INVENTION

The present invention provides methods to blocks the toxic effects of ATP and/or adenosine in the circulation of individuals at risk of dementia or central nervous system (CNS) compromise. In addition, the present disclosure further provides methods for identifying agents that prevent or reduce ATP concentrations in the circulation.

In one embodiment, the present invention includes a method to treat or prevent events of CNS compromise in several neurodegenerative diseases in an individual. The main method is comprising the administration to an individual a compound, peptide, and/or nucleic acid expression vector to block pannexin-1 opening, secretion or stability of ATP, or activation of purinergic receptors to prevent or revert CNS compromise. In another aspect, the nucleic acid expression vector is virus-based. In another aspect, a polypeptide or compound that could target the CNS under a specific or unspecific control element. In another aspect, the pannexin-1 polypeptide, ATP regulator, or purinergic blockers comprises an amino acid sequence or structure having at least 85% identity to the cited blockers. In another aspect, the pannexin-1, ATP enzyme, or purinergic peptides comprises an amino acid sequence having at least about 95% amino acid sequence identity to the original sequence cited above. In another aspect, the neurodegenerative disease to target can be NeuroHIV, Alzheimer's, Parkinson, ALS, MS, and other CNS diseases with high circulating levels of ATP. In another aspect, the individual is a human. In another aspect, a systemic or localized administration of the treatment.

In another embodiment a single or combined method to decrease pannexin-1 channel opening, ATP stability, and purinergic receptor activation using multiple ways described above. In another aspect, targeting the circulation and cells exposed to the circulation. In another aspect, the expression vector is virus-based, peptide, or compound affecting claims 1 and 9. In another aspect, to prevent BBB overactivation and CNS compromise.

In another embodiment, a method for identifying a candidate or candidate agents for the treatment or prevention of neurodegenerative diseases based on ATP dysregulation. The method involves determinations of ATP circulating levels in correlation with cognitive impairment, determining the effect, if any, of a test agent on BBB function, immune activation, inflammation, and CNS compromise, administering a single or combination of treatments to reduce circulating levels of ATP and its effects on a human. In another aspect, the administering is systemic. In another aspect, the expression vectors, peptides, or targeted compounds with one or more agents that facilitate the crossing of the BBB.

In another embodiment a routinary test, to prevent ATP rise in the circulation to maintain the structure and function of the BBB to prevent CNS compromise.

In another embodiment the present invention include a method for at least one of: screening, preventing release, accumulation, or measuring signaling associated with high levels of circulating ATP in a neurodegenerative diseases comprising obtaining a sample from a subject, detecting levels of ATP as biomarkers of cognitive disease; and preventing or treating the subject with effective amount of at least one of: a compound, a peptide, a protein, or a nucleic acid expression vector that expresses a peptide or protein that blocks pannexin-1 opening, secretion or stability of an ATP regulator, or activation of purinergic receptors sufficient to treat or prevent the neurodegenerative disease. In one aspect, the nucleic acid expression vector is virus-based. In another aspect, a polypeptide or compound that could target the CNS under a specific or unspecific control element. In another aspect, the pannexin-1 polypeptide, ATP regulator, or purinergic blockers comprises an amino acid sequence or structure having at least 85% identity to the cited blockers. In another aspect, the pannexin-1, ATP enzyme, or purinergic peptides comprises an amino acid sequence having at least about 95% amino acid sequence identity to the original sequence cited above. In another aspect, the neurodegenerative disease to target can be NeuroHIV, Alzheimer's, Parkinson, ALS, MS, and other CNS diseases with high circulating levels of ATP. In another aspect, the individual is a human. In another aspect, a systemic or localized administration of the treatment.

### BRIEF DESCRIPTION OF THE DRAWINGS

For a more complete understanding of the features and advantages of the present invention, reference is now made to the detailed description of the invention along with the accompanying figures and in which:

FIGS. 1A to 1F show PBMCs isolated from uninfected individuals maintain the pannexin-1 channels in a closed stage and PBMCs isolated from HIV-infected individuals have a spontaneous opening of pannexin-1 channels.

FIGS. 2A to 2F show opening of Pannexin-1 channels on PBMCs is associated with increased circulating levels of PGE2 and ATP in the plasma/serum of HIV-infected individuals.

FIGS. 3A to 3D show PBMCs isolated from HIV-infected individuals release ATP and PGE2 in a pannexin-1 dependent, but not Cx43 hemichannel, manner.

FIGS. 4A to 4D show transmigration uninfected and HIV-infected PBMCs (HIVADA) is pannexin-1 dependent, and secreted ATP contributes to BBB disruption.

FIG. 5 is a graph that shows mRNA expression profile of human primary astrocytes, a key component of the human Blood Brain barrier. qRT-PCR determined that P2X4, P2X7, and P2Y1 are the main expressed purinergic receptor in astrocytes. The negative control, PECAM-1 and the positive control Connexin 43, Cx43, are the internal controls. All other receptors were negatives. N=4 different individuals.

FIG. 6 is a graph that shows mRNA expression of ecto-ATPases on human primary astrocytes, a key component of the blood brain barrier. qRT-PCR determined that NNP1, NNP2, NTPDase 1 and CD73 are the main ecto ATPases expressed in astrocytes. The negative control, PECAM-1 and the positive control Connexin 43, Cx43, are the internal controls. All other receptors were negatives. N=4 different individuals.

FIG. 7 is a graph that shows mRNA expression of adenosine receptors on human primary astrocytes, a key component of the blood brain barrier. qRT-PCR determined that A2A was the only adenosine receptor expressed in astrocytes. The negative control, PECAM-1 and the positive control Connexin 43, Cx43, are the internal controls. All other receptors were negatives. N=4 different individuals.

FIG. 8 is a graph that shows mRNA expression of cell to cell communication mRNA required for efficient astrocyte-astrocyte communication. qRT-PCR determined that Cx43, pannexin-1 and ZO-1 are expressed in astrocytes. The negative control, PECAM-1 and the positive control Connexin 43, Cx43, are the internal controls. All other receptors were negatives. N=4 different individuals.

FIG. 9 is a graph that shows mRNA expression profile of human primary brain endothelial cells, a key component of the human Blood Brain barrier. qRT-PCR determined that P2Y1, Y2, Y4, Y5, Y6, and Y8 are the main expressed purinergic receptor in astrocytes. The positive control, PECAM-1 and the positive control MGMT are the internal controls. All other receptors were negatives. N=4 different individuals.

FIG. 10 is a graph that shows mRNA expression of ecto-ATPases on human primary brain endothelial cells, a key component of the blood brain barrier. qRT-PCR determined that NNP1, NTPDase 1, NTPDase 5 and CD73 as well ADA are the main ecto ATPases expressed in human brain endothelial cells. The positive control, PECAM-1 and the positive control MGMT are the internal controls. All other receptors were negatives. N=4 different individuals.

FIG. 11 is a graph that shows mRNA expression of adenosine receptors on human primary brain endothelial cells, a key component of the blood brain barrier. qRT-PCR determined that A2B was the only adenosine receptor expressed in human primary brain endothelial cells. The positive control, PECAM-1 and the positive control MGMT are the internal controls. All other receptors were negatives. N=4 different individuals.

FIG. 12 is a graph that shows mRNA expression of cell to cell communication mRNA required for efficient endothelial cell communication. qRT-PCR determined that Cx43, pannexin-1 and ZO-1 are expressed in on human primary brain endothelial cells. The negative control, PECAM-1 and the positive control Connexin 43, Cx43, are the internal controls. All other receptors were negatives. N=4 different individuals.

FIG. 13 is a graph that shows mRNA expression profile of human primary astrocytes, a key component of the human Blood Brain barrier, after HIV infection. qRT-PCR determined that P2X1, P2X2, P2X3, P2X4, P2X5, P2X6 and P2X7 are affected by HIV infection. The negative control, PECAM-1 and the positive control Connexin 43, Cx43, are the internal controls. All other receptors were negatives. N=4 different individuals.

FIG. 14 is a graph that shows mRNA expression profile of human primary astrocytes, a key component of the human Blood Brain barrier, after HIV infection. qRT-PCR determined that P2Y1, Y2, Y4, Y5, Y6, Y8, Y10. Y11, Y12, Y13, and Y14 are altered by HIV infection. The negative control, PECAM-1 and the positive control Connexin 43, Cx43, are the internal controls. All other receptors were negatives. N=4 different individuals.

FIG. 15 is a graph that shows mRNA expression of ecto-ATPases on human primary brain endothelial cells, a key component of the blood brain barrier. qRT-PCR determined that several ecto-ATPases are altered by HIV infection. The positive control, PECAM-1 and the positive control MGMT are the internal controls. All other receptors were negatives. N=4 different individuals.

FIG. 16 is a graph that shows mRNA expression of adenosine receptors on human primary brain endothelial cells, a key component of the blood brain barrier. qRT-PCR determined that several adenosine receptors are altered by HIV infection. The positive control, PECAM-1 and the positive control MGMT are the internal controls. All other receptors were negatives. N=4 different individuals.

FIG. 17 is a graph that shows mRNA expression of communication systems on human primary brain endothelial cells, a key component of the blood brain barrier, are altered upon infection. qRT-PCR determined that several communication systems are altered by HIV infection. The positive control, PECAM-1 and the positive control MGMT are the internal controls. All other receptors were negatives. N=4 different individuals.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The inventors recently identified that the ATP system is compromised before the onset of central nervous system (CNS) damage. It was found that ATP is elevated in the circulation of individuals at risk of CNS complications, and ATP levels can predict CNS damage, but also ATP and its metabolites are critical to compromise the blood-brain barrier (BBB) and the CNS. The inventors identify that the uncontrolled opening of pannexin-1 channels mediated ATP into the circulation, and also identified the profile of ATP and adenosine receptors at the BBB that can be targeted to prevent and reduce the consequences of dementia.

As used herein, the terms “polypeptide,” “peptide,” and “protein,” used interchangeably herein, refer to a polymeric form of amino acids of any length, which can include genetically coded and non-genetically coded amino acids, chemically or biochemically modified or derivatized amino acids, and polypeptides having modified peptide backbones as well as with any stabilization method.

As used herein, the terms “nucleic acid” and “polynucleotide” are used interchangeably herein and refer to a polymeric form of nucleotides of any length, either deoxyribonucleotides or ribonucleotides or analogs thereof. Non-limiting examples of polynucleotides include linear and circular nucleic acids, messenger RNA (mRNA), cDNA, recombinant polynucleotides, vectors, probes, and primers.

As used herein, the term “operably linked” refers to a functional linkage between molecules to provide the desired function

As used herein, the terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc. In some embodiments, the individual is human. In some embodiments, the individual is murine, macaques, or rabbit.

As used herein, a “therapeutically effective amount” or “efficacious amount” of a peptide or nucleic acid means the amount of nucleic acid that, when administered to a mammal or other subject for treating a disease, is sufficient to effect such treatment for the disease.

As used herein, the terms, ATP and sub-products, pannexin/connexin hemichannels, purinergic/adenosine receptors and ecto-ATPases are used interchangeably herein to ATP related signaling.

As used herein, the term “determining” refers to both quantitative and qualitative determinations, and as such, the term “determining” is used interchangeably herein with “assaying,” “measuring,” and the like.

As used herein, the terms “candidate agent,” “test agent,” “agent,” “substance,” and “compound” are used interchangeably herein. Candidate agents encompass numerous chemical classes, typically synthetic, semi-synthetic, or naturally occurring inorganic or organic molecules. Candidate agents include those found in large libraries of synthetic or natural compounds. For example, synthetic compound libraries are commercially available from Maybridge Chemical Co. (Trevillet, Cornwall, UK), ComGenex (South San Francisco, Calif.), and MicroSource (New Milford, Conn.). A rare chemical library is available from Aldrich (Milwaukee, Wis.). Alternatively, libraries of natural compounds in the form of bacterial, fungal, plant, and animal extracts are available from Pan Labs (Bothell, Wash.) or are readily producible.

Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); not, nucleotide(s); i.m., intramuscular(ly); i.p., intraperitoneal(ly); s.c., subcutaneous(ly); and the like.

Candidate agents may be small organic or inorganic compounds having a molecular weight of more than 50 and less than about 2,500 daltons. Unconjugated and conjugated peptides also will be tested. Candidate agents may comprise functional groups necessary for structural interaction with other macromolecules such as proteins, e.g., hydrogen bonding, and may include at least an amine, carbonyl, hydroxyl or carboxyl group, and may contain at least two of the functional chemical groups. The candidate agents may comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

A test agent can be a small molecule. The test molecules may be individual small molecules of choice or in some cases, the small molecule test agents to be screened come from a combinatorial library, i.e., a collection of diverse chemical compounds generated by either chemical synthesis or biological synthesis by combining a number of chemicals “building blocks.” For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. Indeed, theoretically, the systematic, combinatorial mixture of 100 interchangeable chemical building blocks results in the synthesis of 100 million tetrameric compounds or 10 billion pentameric compounds. See, e.g., Gallop et al., (1994), J. Med. Chem., 37(9), 1233-1251. Preparation and screening of combinatorial chemical libraries are well known in the art. Combinatorial chemical libraries include but are not limited to diversomers such as hydantoins, benzodiazepines, and dipeptides, as described in, e.g., Hobbs et al., (1993), Proc. Natl. Acad. Sci. U.S.A., 90:6909-6913; analogous organic syntheses of small compound libraries, as described in Chen et al., (1994), J. Amer. Chem. Soc., 116:2661-2662; Oligocarbamates, as described in Cho, et al., (1993), Science, 261:1303-1305; peptidyl phosphonates, as described in Campbell et al., (1994), J. Org. Chem., 59: 658-660; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514).

Numerous combinatorial libraries are commercially available from, e.g., ComGenex (Princeton, N.J.); Asinex (Moscow, Russia); Tripos, Inc. (St. Louis, Mo.); ChemStar, Ltd. (Moscow, Russia); 3D Pharmaceuticals (Exton, Pa.); and Martek Biosciences (Columbia, Md.).

The analysis of known and unknown drugs can be performed using artificial intelligence or other methods to generate a matrix to analyze multiple layers of data.

To clarify the invention described below, it is to be understood that this invention is not limited to a particular paradigm described, as such may, of course, vary. It is also to be understood that the terminology used is to describe particular embodiments only, and it is not intended to be limiting since the scope of the present invention will be discussed in the claims.

Despite that a range of values is provided, it is understood that there is variation in different individuals, and any particular variation is encompassed within the invention.

Unless defined otherwise, all technical and scientific terminology used herein has the same meaning as commonly understood by one ordinary skill in the art to which this invention belongs. Any method and materials similar or equivalent to those described herein can also be used in the practice or testing of the present invention. Publications are cited as references.

It must be noted that as used herein and in the appended claims, the singular forms include plural references unless the context dictates otherwise by including terminology as only or solely.

The publications discussed herein are provided solely for their disclosure prior to the filing date of the present application. Nothing herein is to be construed as an admission that the present invention is not entitled to antedate such publication by virtue of prior invention.

The present disclosure provides a method to reduce or prevent the toxic effect of ATP/ADP/AMP in the circulation as well as increase the action of adenosine in the circulation. In addition, the inventors described several methods of treating an ongoing neurodegenerative disease in an individual. The present disclosure further provides means of identifying an agent or combination of agents to increase the level and/or function of the equilibrium among ATP/ADP/AMP and adenosine.

### Example 1

Methods to detect and intervene in the ATP/ADP/AMP and adenosine pathways to prevent the onset of CNS compromise and cognitive disease. The present disclosure provides several methods to detect, quantify, and to create a custom treatment to block, stimulate or reach an equilibrium among ATP and its sub-products to prevent and reduce the devastating consequences of dementia and associated cognitive disease. The method can be divided into several sections:
Detection and quantification of ATP and its sub-products as a marker of CNS compromise and the basis to generate a treatment to prevent brain damage;
A treatment to prevent ATP secretion by targeting pannexin channels;
A treatment, compound(s) or enzymatic, to reduce the high levels of ATP associated with CNS damage:
Detection and treatment to increase adenosine levels;
A blocking or stimulating compounds, targeting ATP and its sub-product receptors, to prevent endothelial and astrocyte activation; and/or
A combination of the treatments indicated above to prevent high circulating levels or ATP and its sub-products and to block these receptors, transporters, and enzymes before and during blood-brain barrier exposure to prevent and reduce CNS damage.

Detection and quantification of ATP and its sub-products as a marker of CNS compromise and the basis to prevent or reduce associated damage. As the inventors recently described [1], the detection of circulating levels of ATP and its sub-products could be a biomarker of CNS compromise.

ATP and subproduct quantification can be used to predict CNS compromise using. enzymatic activity assays, biotin mediated assays, competition experiments assays, HPLC based assays, mass spectrometry assays, bioluminescence and other assays known in the art.

All these assays need to evaluate circulating amounts of ATP. Still, the inclusion of sub-products can provide further information about the rate of ATP degradation and sub-products.

The present disclosure provides a method of preventing or reduce circulating levels of ATP and increase adenosine to improve endothelial and glial function. A subject method generally involves the treatment with selected compounds (see below, agonist or antagonists), peptides (see below), and/or an exogenous nucleic acid (see below) to produce the effects described above.

A subject method can be used to treat the onset of CNS or PNS damage in the context of HIV, Alzheimer's, MS, ALS, and Parkinson's disease and other neurodegenerative diseases in an individual with ATP/ADP/AMP/adenosine compromise.

It was discovered that ATP and its sub-products are released as an inflammatory factor mainly by the opening of pannexin/connexin hemichannels and contribute to cell death. The inventors generated a unique map of purinergic receptors, ecto-ATPases, and adenosine receptors on human primary brain microvascular endothelial cells as well as astrocytes and pericytes. Several of these receptors changed upon disease conditions, as demonstrated hereinbelow. Thus, personalized medicine, depending on the disease, can be provided.

As noted above, a subject method of treating dementia-related neurodegenerative disease in an individual generally involves administering to the individual an adequate amount of exogenous selected compounds (see below, agonist or antagonists), peptides (see below), and/or an exogenous nucleic acid (see below). A subject method reduces circulating levels of ATP, receptor activation, or preventing the release of ATP and sub-products. “In some embodiments, an “effective amount” of an exogenous treatment is an amount or combination of treatments that are effective in preventing decline cognitive function in an individual. In some embodiments, an “effective amount” of an exogenous treatment is an amount that is effective to reduce or ameliorate one or more adverse symptoms of an onset of CNS induced neurodegenerative disease in an individual.

In some embodiments, an effective amount of these exogenous treatments is an amount that reduces an adverse symptom, abnormality, or pathologies associated with neurodegenerative disease, such as blood-brain disruption, neuronal/glial compromise, viral reactivation in the case of HIV, changes in pathology, pain, reduced/prevented inflammation, or cognitive assessment by at least about 10%, at least about 20%, at least about 30%, at least about 50%, at least about 60%, at least about 70%, at least about 80%, at least about 90% or more. In other embodiments, an effective amount of treatment is an amount or combination of treatments that improve a parameter that is in decline in individuals with neurodegenerative diseases, such as blood-brain disruption, neuronal/glial compromise, viral reactivation in the case of HIV, changes in pathology, pain, reduced/prevented inflammation, or cognitive assessment, by at least about 10%, at least about 20%, at least about 30%, at least about 50%, at least about 60%, at least about 70%, at least about 80%, at least about 90% or more, such that the decline in one of these parameters is at least slowed.

### Example 2

Blocking pannexin/connexin hemichannels to reduce ATP and their sub-product secretion. It has been shown in several neurological diseases indicated that pannexin-1 channels become open, resulting in the release of several intracellular second messengers that amplify inflammation, blood-brain barrier disruption, and CNS compromise. Thus, blocking the opening of pannexin channels with siRNA, peptides, or chemicals (probenecid and others) can reduce and prevent the toxic effects of pannexin-1 channel opening (see table 1).

TABLE 1
Pannexin-1 blockers

| | | Used in |
|---|---|---|
| Compound family | Target | Humans |
| Carbenoxolone | Panx-1 | Yes, gastric ulcer |
| Flufenamic acid | Panx-1 | Yes, it reduces platelet |
| | | aggregation. |
| | | COX mechanism |
| Probenecid | Panx-1 | Yes, gout treatment |
| Brilliant Blue FCF | Panx-1 | No |
| 10Panx-1 peptide | Panx-1 | No |
| Tat-Panx-1308 | Panx-1 | No |
| siRNA or similar to Panx-1 | Panx-1 | No |
| Tenofovir | Panx-1 | Yes, liver and skin fibrosis |
| Trovafloxacin mesylate | Panx-1 | Yes, antibiotic |
| Mefloquine | Panx-1 | Yes, antimalarial |

In some embodiments, a polypeptide treatment comprises an amino acid sequence having at least about 80%, at least about 85%, at least about 90%, at least about 95%, at least about 98%, at least about 99%, or 100%6, amino acid sequence identity to a contiguous stretch of from about 8, 9, 10, 15, 20, 25, 30, 35 to 40 amino acids (aa).

In some embodiments, a polypeptide comprises an amino acid sequence having at least about 80%, at least about 85%, at least about 90%, at least about 95%, at least about 98%, at least about 99%, or 100%, amino acid sequence identity to a contiguous stretch of from about 50, 60, 70, 75 80, 90 to 100 amino acids (aa) depending on the proteins targeted.

Exogenous pannexin/connexin peptides alone or conjugated nucleic acid can be a recombinant expression vector, where suitable vectors include, e.g., recombinant retroviruses, lentiviruses, and adenoviruses; retroviral expression vectors, lentiviral expression vectors, nucleic acid expression vectors, and plasmid expression vectors. In some cases, the exogenous nucleic acid is integrated into the host genome. In other cases, the exogenous nucleic acid persists in an episomal state. In some cases, an endogenous, a natural version of an exogenous nucleic acid exists in the cells; and the introduction of the exogenous nucleic acid increases the level and/or function of ATP/adenosine receptors in the cell. In other cases, the exogenous nucleic acid encodes a pannexin/connexin polypeptide having an amino acid sequence that differs by one or more amino acids from an extracellular portion of the polypeptide encoded by an endogenous encoding nucleic acid within the host cell.

Suitable expression vectors include, but are not limited to, viral vectors (e.g., viral vectors based on vaccinia virus, poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641648, 1999; Ali et al., Hum Mol Genet. 5:591594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like, relevant portions incorporated herein by reference.

Numerous suitable expression vectors are known to those of skill in the art, and many are commercially available (see Table 1). For eukaryotic host cells, the following vectors are provided by way of example: pXT1, pSG5 (Stratagene), pSVK3, pBPV, pMSG, and pSVLSV40 (Pharmacia). However, any other vector may be used so long as it is compatible with the host cell and can deliver the load into targeted cells or be sustainable for personalized medicine.

Depending on the host/vector system utilized, any of a number of suitable transcription and translation control elements, including constitutive and inducible promoters, transcription enhancer elements, transcription terminators, etc. may be used in the expression vector (see, e.g., Bitter et al. (1987) Methods in Enzymology, 153:516-544), relevant portions incorporated herein by reference.

In some embodiments, an encoding nucleotide sequence is operably linked to a control element, e.g., a transcriptional control element, such as a promoter. The transcriptional control element is functional in a eukaryotic cell, e.g., a mammalian cell. Suitable transcriptional control elements include promoters and enhancers. In some embodiments, the promoter is constitutively active. In other embodiments, the promoter is inducible.

Non-limiting examples of suitable eukaryotic promoters (promoters functional in a eukaryotic cell) include CMV immediate early, HSV thymidine kinase, early and late SV40, long terminal repeats (LTRs) from retrovirus, and mouse metallothionein-I.

In some embodiments, the encoding nucleotide sequence is operably linked to a cell-specific control element (e.g., a promoter, an enhancer), a microglia-specific transcriptional control element, an oligocyte-specific transcriptional control element, or an astroglia-specific transcriptional control element. Selection of the appropriate vector and promoter is well within the level of ordinary skill in the art. The expression vector may also contain a ribosome binding site for translation initiation and a transcription terminator. The expression vector may also include appropriate sequences for amplifying expression.

Methods of producing recombinant viruses from packaging cells and their uses are well established; see, e.g., U.S. Pat. Nos. 5,834,256; 6,910,434; 5,591,624; 5,817,491; 7,070,994; and 6,995,009. Many methods begin with the introduction of a viral construct into a packaging cell line, relevant portions incorporated herein by reference. The viral construct may be introduced into a host fibroblast by any method known in the art, including but not limited to: a calcium phosphate method, a lipofection method (Feigner et al. (1987) Proc. Natl. Acad. Sci. U.S.A. 84:7413-7417), an electroporation method, microinjection, Fugene transfection, and the like, and any method described herein, relevant portions incorporated herein by reference.

A nucleic acid construct can be introduced into a host cell using a variety of well-known techniques, such as non-viral based transfection of the cell. In an exemplary aspect, the construct is incorporated into a vector and introduced into a host cell. Introduction into the cell may be performed by any non-viral based transfection known in the art, such as but not limited to, electroporation, calcium phosphate mediated transfer, nucleofection, sonoporation, heat shock, magnetofection, liposome-mediated transfer, microinjection, microprojectile mediated transfer (nanoparticles), cationic polymer mediated transfer (DEAE-dextran, polyethylenimine, polyethylene glycol (PEG) and the like) or cell fusion. Other transfection methods include transfection reagents such as Lipofectamine™, Dojindo Hilymax™, Fugene™, jetPEI™, Effectene™, and DreamFect™ or newest techniques (e.g., mRNA vaccine methods).

### Example 3

Inducing degradation of ATP in the circulation to prevent blood-brain barrier and nervous system compromise. The data shows that circulating levels of ATP increased in correlation with signs and symptoms of brain damage and cognitive disease. The inventors demonstrated that levels of circulating ATP compromise BBB and cognition. Thus, reducing the levels of ATP can prevent BBB disruption and neuronal/glial compromise. Currently, the treatments to speed up ATP degradation or alternatively blocking ATP synthesis via ATP synthase inhibition are related to bacterial/fungal products as well as recombinant enzymes that can reduce circulating levels of ATP. More than 250 natural and synthetic inhibitors have been classified to date, with reports of their known or proposed inhibitory sites and modes of action. Selected ATP synthase inhibitors are shown in Table 2. ATP is a crucial factor in inflammation, cell differentiation, tumorigenesis, and danger signal [7-21]. Thus, reducing the level of ATP can have multiple clinical applications.

TABLE 2
Selected Inhibitors of ATP Synthase

| | | Used in |
|---|---|---|
| Compound Family | Target | Humans |
| Diarylquinoline (TM207, | ATP Synthase | Y, antimycobacterial |
| Bedaquiline) | | |
| Oligomycin A | ATP Synthase | Y, antibiotic |
| Melittin | ATP Synthase | Y, anti-inflammatory |
| Polyphenols (e.g. quercetin, | ATP Synthase | N |
| resveratrol) | | |
| BZ-423 | ATP Synthase | Y, autoimmune disease |

Exogenous bacterial/fungal products, and recombinant enzymes, can be produced through recombinant expression vectors. Suitable vectors include, recombinant retroviruses, lentiviruses, and adenoviruses; retroviral expression vectors, lentiviral expression vectors, nucleic acid expression vectors, and plasmid expression vectors. In some cases, the exogenous nucleic acid is integrated into the host genome. In other cases, the exogenous nucleic acid persists in an episomal state. In some cases, an endogenous, a natural version of an exogenous nucleic acid exists in the cells; and the introduction of the exogenous nucleic acid increases the level and/or function of enzymes that degrade ATP. In other cases, the exogenous nucleic acid encodes an enzyme or protein that accelerates ATP degradation having an amino acid sequence that differs by one or more amino acids from an extracellular portion of the polypeptide encoded by an endogenous encoding nucleic acid within the host cell.

### Example 4

Blocking purinergic receptors and/or activation of adenosine receptors to prevent nervous system compromise. The data in several neurological diseases showed that ATP accumulates and circulates in the blood for extended periods resulting in BBB compromise and CNS dysfunction. Normal and overloaded ATP signaling is mediated by the combination of receptors for ATP, ADP, AMP, and adenosine. These data using human primary endothelial cells demonstrates that purinergic receptors are highly expressed on brain endothelial cells as compared to other CNS cell types, suggesting a strong response upon ATP. Thus, the inventors prevented the activation of these purinergic receptors using chemical and siRNA.

Exogenous chemicals and siRNA alone or conjugated nucleic acid can be a recombinant expression vector, where suitable vectors include, e.g., recombinant retroviruses, lentiviruses, and adenoviruses; retroviral expression vectors, lentiviral expression vectors, nucleic acid expression vectors, and plasmid expression vectors. In some cases, the exogenous nucleic acid is integrated into the host genome. In other cases, the exogenous nucleic acid persists in an episomal state. In some cases, an endogenous, a natural version of an exogenous nucleic acid exists in the cells; and the introduction of the exogenous nucleic acid increases the level and/or function of ATP/adenosine receptors in the cell. In other cases, the exogenous nucleic acid encodes a purinergic receptor having an amino acid sequence that differs by one or more amino acids from an extracellular portion of the polypeptide encoded by an endogenous encoding nucleic acid within the host cell.

TABLE 3
purinergic blockers

| | | Used in |
|---|---|---|
| Compound family | Target | Humans |
| PSB family | Purinergic allosteric, P2X1, P2X2 | No |
| Bx-430 | Non competitive, P2X1, P2X7 | No |
| Carbamazepine | Negative allosteric, P2X1, P2X2 | No |
| NP-1815 | No reported, P2X1 | No |
| Ivermectin | Not reported | No |
| PPADS | Not reported | No |
| MRS2500 | Not reported | No |
| Suramin | Not reported | No |
| NF compounds | Not reported | No |
| AR-compounds | | No |
| A3p5p | | No |
| TNP-ATP | P2X1 | No |
| NF023 compounds | P2X1 | No |
| KN62/04 | P2X7 | No |
| Apyrase | ATP degradation | No |

Formulations, Dosages, and Routes of Administration. As discussed above, a subject treatment method generally involves administering to an individual in need thereof a sufficient amount of compounds, peptides, or an exogenous nucleic acid to regulate circulating levels of ATP/ADP/AMP/adenosine. Formulations, dosages, and routes of administration are discussed below. For the discussion of formulations, dosages, and routes of administration, the term “active agent” refers to a treatment to target circulating levels of ATP/ADP/AMP/adenosine. In some instances, a composition comprising an active agent can comprise a pharmaceutically acceptable excipient, a variety of which are known in the art and need not be discussed in detail herein. Pharmaceutically acceptable excipients have been amply described in a variety of publications.

Pharmaceutical compositions can be formulated for controlled or sustained delivery in a manner that provides a local concentration of an active agent (e.g., bolus, depot effect, capsule) and/or increased stability or half-life in a particular local environment. The compositions can include the formulation of exogenous drugs or nucleic acids with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then can be delivered as a depot injection. Techniques for formulating such sustained- or controlled-delivery means are known, and various polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH-sensitive properties, may be desirable for the drug, virus, or peptide depot effect because of the mild and aqueous conditions involved in trapping an active agent.

The formulation can contain components in addition to nucleic acid, where the aid of the additional component in the delivery of the nucleic acid. The nucleic acid can be present in a pharmaceutical composition with other elements such as, but not limited to, stabilizing compounds and/or biocompatible pharmaceutical-carriers, e.g., saline, buffered saline, dextrose, or water. The nucleic acid can also be administered alone or in combination with other agents, including other therapeutic agents. The formulation can also contain organic and inorganic compounds to, for example, facilitate nucleic acid delivery to and uptake by the target cell (e.g., detergents, salts, chelating agents, etc.).

The nucleic acid or drug formulation is administered orally. The formulation can contain buffering agents or a coating to protect the nucleic acid from stomach acidity and/or facilitate swallowing. In addition, the oral formulation can be administered during an interdigestive period (between meals or at bedtime) when stomach pH is less acidic, or with the administration of inhibitors of acid secretion such as H2 blockers (e.g., cimetidine) or proton pump inhibitors (e.g., PROLISEC™) The formulation can also comprise a time-release capsule designed to release the nucleic acid upon reaching the surface of intestinal cells.

Dosage levels can be readily determined by the ordinarily skilled clinician and can be modified as required, e.g., as needed to achieve the desired effect. Dosage levels can be on the order of about 0.1 mg to about 100 mg per kilogram of body weight per day. The amount of active agent (DNA or compounds) combined with the carrier materials to produce a single dosage form varies depending upon, e.g., the host treated and the particular mode of administration. Dosage unit forms can contain between about 1 mg to about 500 mg of an active agent.

An active agent can be delivered via any of a variety of modes and routes of administration, including, e.g., local delivery by injection; local delivery by continuous release; systemic delivery by oral administration; systemic delivery by intravenous administration; and the like. An active agent can be delivered by various routes, including intracranial, intrathecal, intraventricular, intracapsular, and other routes of administration.

In another embodiment, a controlled release system can be placed in proximity to the target tissue. For example, a micropump may deliver controlled doses directly into the brain, thereby requiring only a fraction of the systemic dose (see, e.g., Goodson, 1984, in Medical Applications of Controlled Release, vol. 2, pp. 115-138).

In one embodiment, it may be desirable to administer the agent locally to the area in need of treatment; this may be achieved, for example, and not by way of limitation, by local infusion during surgery, topical application, injection, by means of a catheter, by means of a suppository, or by means of an implant. An implant can be porous, non-porous, or gelatinous material, including membranes, such as sialastic membranes, or fibers.

Several methods can achieve the delivery of therapeutic agents to the CNS. In some embodiments, a DNA or compound(s) will be formulated and/or delivered in such a way as to facilitate or bypass crossing the blood-brain barrier (BBB). Molecules that cross the blood-brain barrier use two main mechanisms: free diffusion; and facilitated transport. These data validate a unique screening of primary human cells to prevent BBB compromise and give a better target to reduce the devastating consequences of CNS damage.

Administration can be systemic or local. In some embodiments, a DNA or compound(s) is introduced into the central nervous system by any suitable route, including intraventricular and intrathecal injection; for example, an intraventricular injection may be facilitated by an intraventricular catheter attached to a reservoir, such as an Ommaya reservoir.

Combination Therapies. In some embodiments, a subject method further includes administering at least one additional therapeutic agent. Suitable additional therapeutic agents include, but are not limited to, non-steroidal anti-inflammatory agents, including, but not limited to, ibuprofen and indomethacin; cyclooxygenase-2 (Cox2) inhibitors such as Celebrex; and monoamine oxidase inhibitors, such as Selegiline (Eldepryl or Deprenyl). Dosages for each of the above agents are known in the art. For example, Aricept is generally administered at 50 mg orally per day for six weeks, and, if well tolerated by the individual, at 10 mg per day after that.

Subjects Suitable for Treatment. Subjects suitable for treatment with a method of treating neurodegenerative disorder include individuals who have been diagnosed as having a disorder such as neuroHIV, Alzheimer's disease, Parkinson's disease, ALS, MS, age-related dementia, cerebral or systemic amyloidosis, hereditary cerebral hemorrhage with amyloidosis, or Down's syndrome.

Screening Methods. The present disclosure provides methods of identifying a candidate agent for the treatment of neurodegenerative diseases. In some embodiments, the methods generally involve a) administering a unique or combination of DNA or compounds to reduce circulating levels of ATP and the activation of purinergic receptors within the CNS to block or prevent nervous system damage, and b) determining the effect, if any, of the test agent on preventing opening of pannexin-1 channels. A test agent that reduces ATP, pannexin-1 channel opening, or blocks purinergic receptor activation is a candidate agent for treating neurodegenerative diseases. In other embodiments, the methods generally involve a) contacting a cell that expresses pannexin channels or ATP/adenosine receptors with a test agent; and b) determining the effect, if any, of the test agent on the level of activation of the pannexin channels or ATP/adenosine receptors in cells. A test agent that blocks the opening of pannexin channels or activation of ATP/adenosine receptors in the cell is a candidate agent for treating neurodegenerative diseases. In some embodiments, the cell is a neuron, glia, endothelial, or transfected cell.

Assays of the invention include controls, where suitable controls include a sample in the absence of the test agent. Generally, a plurality of assay mixtures is run in parallel with different test agent concentrations to obtain a differential response to the various concentrations. Typically, one of these concentrations serves as a negative control, i.e., at zero concentration or below the level of detection.

A variety of other reagents may be included in the screening assay. These include reagents such as salts, neutral proteins, e.g., albumin, detergents, etc., that are used to facilitate optimal protein-protein binding and/or reduce non-specific or background interactions. Reagents that improve the efficiency of the assay, such as protease inhibitors, nuclease inhibitors, anti-microbial agents, etc., may be used. The components of the assay mixture are added in any order that provides for the requisite binding or other activity. Incubations are performed at any suitable temperature, typically between 4° C. and 40° C. Incubation periods are selected for optimum activity, but may also be optimized to facilitate rapid high-throughput screening. Usually, between 0.1 and 1 hour will be sufficient.

Cell-Free In Vitro Assays. To identify potential targets to prevent the toxic effects of ATP, in these embodiments, the assay is a cell-free in vitro assay.

A test agent that reduces the opening of pannexin channels, activation of ATP receptors and calibration of the ratio of ATP/adenosine by at least about 5%, at least about 10%, at least about 15%, at least about 20%, at least about 25%, at least about 30%, at least about 35%, at least about 40%, at least about 50%, or more than 50%, compared to the level of binding of the drug/peptide/virus in the absence of the test agent, is a candidate agent for treating neurodegenerative disease.

For example, pannexin targeted polypeptides are described. The pannexin polypeptide can also comprise a moiety that provides for detection, purification, or immunoprecipitation, e.g., a radiolabel, biotin, a fluorescent protein, (His)n, (e.g., 6His), glutathione-S-transferase (GST), hemagglutinin (HA; e.g., CYPYDVPDYA; SEQ ID NO:6), FLAG (e.g., DYKDDDDK; SEQ ID NO.7), c-myc (e.g., CEQKLISEEDL; SEQ ID NO. 8), immunoglobulin Fc, and the like.

The effect of a test agent can be determined using any known assay for assessing the binding of one polypeptide to another and channel opening. Examples include, e.g., an enzyme-linked immunosorbent assay, an immunoprecipitation assay, and the like.

A test agent of interest is assessed for any cytotoxic activity (other than antiproliferative activity) it may exhibit toward a living eukaryotic cell, using well-known assays, such as trypan blue dye exclusion, an MTT (3-(4,5-dimethylthiazol-2-yl)-2,5-diphenyl-2H-tetrazolium bromide) assay, and the like. Agents that do not exhibit cytotoxic activity may be candidate agents for use in a treatment method.

Cell-Based Assays. A test agent that increases the opening or activation of ATP/adenosine receptor in the cell is a candidate agent for treating neurodegenerative diseases. As noted above, in some embodiments, a subject method generally involves a) contacting a cell expressing pannexin channels or ATP/adenosine receptors with a test agent; and b) determining the effect, if any, of the test agent on the level and/or function of pannexin channels or ATP/adenosine receptor activation or blocking in the cell. Such an assay is a cell-based in vitro assay.

The effect of the test agent in the cell can be determined using an immunological assay, e.g., using antibodies or dyes or kinase activation. In some embodiments, the cells (“host cells”) used in the assays are mammalian. Suitable host cells include eukaryotic host cells cultured in vitro, either in suspension or as adherent cells. In some embodiments, the cell is a neuron, glia, macrophages, endothelial cells, or other cell types.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), human embryonic kidney (HEK) cells (ATCC No. CRL1573), HLHepG2 cells, and the like.

Suitable cell lines include, but are not limited to, a human glioma cell line, e.g., SVGp12 (ATCC CRL-8621), CCF-STTG1 (ATCC CRL-1718), SW 1088 (ATCC HTB-12), SW 1783 (ATCC HTB-13), LLN-18 (ATCC CRL-2610), LNZTA3WT4 (ATCC CRL-11543), LNZTA3WT11 (ATCC CRL-11544), U-138 MG (ATCC HTB-16), U-87 MG (ATCC HTB-14), H4 (ATCC HTB-148), and LN-229 (ATCC CRL-2611); a human medulloblastoma-derived cell line, e.g., D342 Med (ATCC HTB-187), Daoy (ATCC HTB-186), D283 Med (ATCC HTB-185); a human tumor-derived neuronal-like cell, e.g., PFSK-1 (ATCC CRL-2060), SK-N-DZ (ATCCCRL-2149), SK-N-AS (ATCC CRL-2137), SK-N-FI (ATCC CRL-2142), IMR-32 (ATCC CCL-127), etc.; a mouse neuronal cell line, e.g., BC3H1 (ATCC CRL-1443), EOC1 (ATCC CRL-2467), C8-D30 (ATCC CRL-2534), C8-S(ATCC CRL-2535), Neuro-2a (ATCC CCL-131), NB41A3 (ATCC CCL-147), SW10 (ATCC CRL-2766), NG108-15 (ATCC HB-12317); a rat neuronal cell line, e.g., PC-12 (ATCC CRL-1721), CTX TNA2 (ATCC CRL-2006), C6 (ATCC CCL-107), F98 (ATCC CRL-2397), RG2 (ATCC CRL-2433), B35 (ATCC CRL-2754), R3 (ATCC CRL-2764), SCP (ATCC CRL-1700), OA1 (ATCC CRL-6538).

The cell used in the assay express pannexin and ATP/adenosine receptors endogenously or transfected. The cell used in the assay can be genetically modified with a recombinant expression vector(s) comprising a nucleotide sequence encoding the system described above in the cell. In general, genetically modified cells can be produced using standard methods. Expression constructs comprising these proteins are introduced into the host cell using standard methods practiced by one with skill in the art. In some embodiments, the polypeptide is encoded on a transient expression vector (e.g., the vector is maintained in an episomal manner by the cell). Alternatively, or in addition, the polypeptide-encoding expression construct can be stably integrated into the cell line.

Behavioral Studies. A candidate agent can be further evaluated for its effect on behavioral parameters, e.g., learning and memory. Behavioral tests designed to assess learning and memory deficits can be employed. An example of the test is the Morris water maze (Morris Learn Motivat 12:239-260 (1981)). In this procedure, the animal is placed in a circular pool filled with water, with an escape platform submerged just below the surface of the water. A visible marker is placed on the platform so that the animal can find it by navigating toward a proximal visual cue. Alternatively, a more sophisticated test in which there are no formal cues to mark the platform's location is given to the animals. In this form, the animal must learn the platform's location relative to distal visual cues. Alternatively, or in addition, memory and learning deficits can be studied using a three-runway panel for working memory impairment (attempts to pass through two incorrect panels of the three panel-gates at four choice points) (Ohno et al. Pharmacol Biochem Behav 57:257-261 (1997)). For HIV studies, humanized mice or animals expressing viral proteins are necessary. For other neurodegenerative diseases, several models are available.

### Example 5

In developed countries, Human Immunodeficiency Virus type-1 (HIV-1) infection has become a chronic disease despite the positive effects of anti-retroviral therapies (ART), but still at least half of the HIV infected population shown signs of cognitive impairment. Therefore, biomarkers of HIV cognitive decline are urgently needed.

The inventors analyzed the opening of one of the larger channels expressed by humans, pannexin-1 channels, in the uninfected and HIV infected population (n=175). Upon opening several intracellular second messengers are released into the extracellular space, including PGE2 and ATP in serum/plasma. The inventors correlated the cognitive status of the patients with circulating levels of both factors upon a visit to the clinic.

Here, it was demonstrated that pannexin channels on fresh PBMCs from uninfected individuals are closed. In contrast, all HIV-infected individuals analyzed, even the ones under effective ART, had a spontaneous opening of pannexin-1 channels and increased circulating levels of PGE2 and ATP. These results suggest that even low or undetectable levels of HIV result in chronic inflammation and opening of pannexin-1 channels. Furthermore, the inventors found that circulating levels of ATP, but not PGE2, correlated with the cognitive status of people living with HIV. Circulating levels of ATP could predict CNS compromise and lead to the breakthroughs necessary to detect and prevent brain compromise in the HIV-infected population. Currently, there are no biomarkers to predict or identify brain compromise in the HIV infected population. HIV-associated neurocognitive disorders (HAND) occurs in at least 50% of the HIV infected population despite effective ART. Commonly, CNS damage in the HIV infected population is assessed by neurophysiological testing batteries and by imaging techniques. For several years, some potential biomarkers in the CSF have been proposed, such as neurofilament (NFL) that is released into the CSF as a result of neuronal damage. Thus, NFL is a late representation of brain damage, and it is not specific for HIV as well as CSF is difficult and painful to obtain. Thus, the necessity for a peripheral CNS biomarker of CNS in the HIV infected population is urgent.

The data identify the mechanism by which a potential biomarker of CNS disease, ATP, is released into the circulation and the impact of this biomarker in blood-brain barrier function, stability, and neuroinflammation. ATP levels in serum were predictive of CNS damage and cognitive decline in the HIV infected population.

Implications of all the available evidence. These results indicate that circulating levels of ATP are useful biomarkers of cognitive disease in the HIV-infected population. By way of explanation, and in no way a limitation of the present invention, the inventors propose that regular determination of circulating levels of ATP can be used to identify individuals under risk of cognitive disease. Furthermore, blocking Pannexin-1 channels or activation of purinergic receptors can generate clinical interventions to prevent CNS damage in the HIV infected population.

The pathogenesis of HIV infection involves a series of dynamic interactions between HIV and several host proteins to support effective HIV infection, replication, generation of viral reservoirs, and associated inflammation [22-24]. Despite effective antiretroviral treatment (ART), most HIV-infected individuals still showed strong evidence of chronic systemic and brain inflammation resulting in cognitive impairment. However, the mechanisms of damage are still elusive.

Recently, the present inventors (and others) identified a novel host protein involved in HIV entry and replication, named pannexin-1 (Panx-1) [25-30]. Pannexin-1 proteins form a large plasma membrane channel that, upon opening, allows the release of several intracellular mediators, including ATP and other nucleotides, prostaglandins, glutamate, NAD+, and metabolites such as glucose. In physiological conditions, these channels remain closed. However, in pathological conditions, including HIV infection, these channels open and amplify inflammation as well as HIV infection and HIV latency/reactivation [29, 31-33. In the context of HIV, the present inventors (and others) identified that the binding of HIV to CD4 receptor and CXCR4 and/or CCR5 co-receptors, induces opening of Panx-1 channels, resulting in local ATP release through the pore and subsequent autocrine and paracrine purinergic receptor activation, which accelerates HIV entry into immune cells [4, 33].

The data demonstrates that panx-1 channels are closed in uninfected individuals, with low to undetectable circulating levels of ATP and PGE2 as expected, second, fresh cells isolated from HIV-infected individuals have spontaneous opening of Panx1 channels, despite the fact that most of the individuals analyzed had low to undetectable HIV replication and normal CD4 counts; third, all HIV-infected individuals analyzed had increased circulating levels of PGE2 and ATP, and both inflammatory compounds were released through the opening of Panx-1 channels; and fourth, circulating ATP levels, but not PGE2, there is a biomarker of HIV cognitive disease. Further, the inventors found that the concentrations of ATP associated with cognitive disease and compromise of the blood-brain barrier (BBB) and increase the transmigration of leukocytes across the BBB in a Panx-1 dependent manner; a critical hallmark of NeuroHIV. These results demonstrate that circulating levels of ATP are useful biomarkers of cognitive disease in the HIV-infected population. Therefore, blocking Panx-1 channels or targeting the circulating ATP provides an excellent therapeutic intervention in all HIV-infected individuals at risk of acquiring cognitive disease and other consequences of HIV chronic associated disease.

Materials and Methods.

Subjects: Serum/plasma samples were obtained from the National NeuroAIDS Tissue Consortium (NNTC) and the CNS HIV Antiretroviral Therapy Effects Research (CHARTER) n=175. All samples had a cognitive assessment and other clinical information available, as described in Table 1. All the analyses were performed blindly.

Neurocognitive examination. NNTC and CHARTER perform a comprehensive neurocognitive test battery every 6 months, including motor function (perceptual-motor speed), verbal fluency, executive function, attention/working memory, speed of information processing, learning, and memory [34-36], for details, see http://www.mountsinai.org/patient-care/service-areas/neurology/areas-of-care/neuroaids-program or https.//nntc.org/.

Fresh blood from local participants. For live-cell imaging experiments, 10 to 15 mL of blood from HIV-seropositive participants were obtained through MHBB, a research resource operating at the Icahn School of Medicine at Mount Sinai (New York, N.Y.), from uninfected volunteers at Rutgers University (Newark, N.J.), or from leukopacks from the NY/NJ Blood Center. HIV-positive individuals were assayed for CD4 cell counts, plasma viral loads and urine toxicology, and underwent neuropsychological testing at the time of blood draw. Patient demographic and virological information is listed in Table 1. Patients gave written, informed consent for the provision of blood for the purposes of HIV research before inclusion in the current CHARTER pilot study. The protocol for blood collection and analysis was approved by the Mount Sinai, Rutgers University and University of Texas Medical Branch Institutional Review Board (Protocol Numbers, Pro20140000794, Pro2012001303, 18-0136, 18-0135, 18-0134).

TABLE 4
Patient Information

| | | | | | CD4 | Viral | Years |
|---|---|---|---|---|---|---|---|
| Patient | HIV | | | Cognitive | counts | Load | with |
| number | status | Age | Gender | assessment | (cells/mm) | (copies/ml) | HIV |
| 1 | + | 30 | M | HAD | 371 | 21085 | 21 |
| 2 | + | 30 | M | MND | 185 | 2544 | 16 |
| 3 | + | 31 | M | N.I. | 129 | 40 | 19 |
| 4 | + | 31 | M | N.I. | 219 | U.D. | 21 |
| 5 | + | 31 | M | N.I. | 108 | 50 | 24 |
| 6 | + | 31 | M | MND | 93 | 1988 | 24 |
| 7 | + | 32 | M | MND | 70 | 1054 | N.R. |
| 8 | + | 32 | M | MND | 18 | 8645 | N.R. |
| 9 | + | 32 | M | MND | 77 | 5365 | 24 |
| 10 | + | 42 | M | MND | 12 | 1005 | 24 |
| 11 | + | 42 | M | HAD | 2 | 10545 | 24 |
| 12 | + | 42 | M | HAD | 25 | 105264 | 9 |
| 13 | + | 43 | M | MND | 31 | 20344 | 9 |
| 14 | + | 43 | M | HAD | 3 | 10256 | 11 |
| 15 | + | 43 | M | HAD | 7 | 8685 | 15 |
| 16 | + | 39 | M | MND | 11 | 2015 | 16 |
| 17 | + | 39 | M | HAD | 4 | 81975 | 4 |
| 18 | + | 39 | M | HAD | 9 | 26759 | 18 |
| 19 | + | 39 | F | MND | 5 | 62357 | 19 |
| 20 | + | 39 | M | HAD | 8 | 20157 | 7 |
| 21 | + | 40 | M | HAD | 4 | 201545 | 7 |
| 22 | + | 40 | F | HAD | 5 | 156841 | 6 |
| 23 | + | 43 | M | MND | 21 | 27951 | 13 |
| 24 | + | 38 | M | N.I. | 185 | 3678 | 11 |
| 25 | + | 38 | M | MND | 64 | 2015 | 7 |
| 26 | + | 39 | M | MND | 70 | 68951 | 12 |
| 27 | + | 39 | M | HAD | 70 | 48001 | 15 |
| 28 | + | 40 | M | N.I. | 81 | 2687 | 10 |
| 29 | + | 41 | M | MND | 74 | 2468 | 9 |
| 30 | + | 42 | M | N.I. | 92 | 50 | 17 |
| 31 | + | 42 | M | MND | 197 | 60257 | N.R. |
| 32 | + | 43 | M | HAD | 86 | 298564 | 15 |
| 33 | + | 43 | F | HAD | 66 | 128978 | 15 |
| 34 | + | 34 | M | MND | 48 | 50004 | 19 |
| 35 | + | 35 | M | MND | 40 | 63904 | 7 |
| 36 | + | 33 | M | HAD | 9 | 3045 | 6 |
| 37 | + | 52 | M | N.I. | 172 | 50 | 6 |
| 38 | + | 43 | F | N.I. | 112 | 50 | 9 |
| 39 | + | 45 | M | MND | 499 | <20 | 14 |
| 40 | + | 50 | M | MND | 334 | <20 | 15 |
| 41 | + | 47 | F | N.I. | 365 | 169 | 10 |
| 42 | + | 52 | F | N.I. | 253 | 2005 | 3 |
| 43 | + | 42 | F | N.I. | 630 | <20 | 5 |
| 44 | + | 40 | F | MND | 473 | U.D. | 7 |
| 45 | + | 52 | F | MND | 299 | U.D. | 12 |
| 46 | + | 49 | F | N.I. | 440 | 50 | 12 |
| 47 | + | 42 | F | N.I. | 241 | 50 | 12 |
| 48 | + | 62 | F | MND | 224 | <20 | 18 |
| 49 | + | 42 | F | MND | 105 | 50 | 14 |
| 50 | + | 37 | F | MND | 312 | <20 | 15 |
| 51 | + | 69 | F | MND | 518 | <20 | 13 |
| 52 | + | 37 | F | N.I. | 231 | 168 | 8 |
| 53 | + | 53 | M | N.I. | 199 | 865 | 21 |
| 54 | + | 57 | M | N.I. | 409 | U.D. | 19 |
| 55 | + | 66 | M | MND | 422 | <20 | 19 |
| 56 | + | 36 | M | N.I. | 510 | U.D. | 10 |
| 57 | + | 78 | F | N.I. | 436 | <20 | 19 |
| 58 | + | 71 | F | N.I. | 560 | U.D. | 18 |
| 59 | + | 62 | F | N.I. | 358 | 265 | 17 |
| 60 | + | 35 | M | N.I. | 300 | 1002 | 14 |
| 61 | + | 52 | M | N.I. | 299 | 2451 | 9 |
| 62 | + | 36 | M | N.I. | 356 | U.D. | 9 |
| 63 | + | 56 | M | MND | 409 | 1024 | 8 |
| 64 | + | 52 | M | N.I. | 504 | U.D. | 24 |
| 65 | + | 83 | M | N.I. | 425 | U.D. | 24 |
| 66 | + | 52 | F | N.I. | 468 | U.D. | 21 |
| 67 | + | 45 | F | MND | 278 | 13645 | 19 |
| 68 | + | 45 | M | N.I. | 308 | <20 | 18 |
| 69 | + | 43 | M | MND | 109 | 199 | 18 |
| 70 | + | 57 | M | N.I. | 323 | U.D. | 13 |
| 71 | + | 58 | M | N.I. | 171 | U.D. | 27 |
| 72 | + | 48 | F | MND | 199 | 20 | 15 |
| 73 | + | 43 | F | HAD | 401 | 81 | 17 |
| 74 | + | 43 | M | N.I. | 392 | U.D. | 21 |
| 75 | + | 32 | F | MND | 185 | 201 | 17 |
| 76 | + | 27 | M | MND | 225 | 99 | 13 |
| 77 | + | 29 | F | N.I. | 407 | U.D. | 12 |
| 78 | + | 41 | M | MND | 399 | N.R. | 8 |
| 79 | + | 41 | F | N.I. | 510 | 20 | 5 |
| 80 | + | 58 | M | NPI-O | 427 | 57 | 7 |
| 81 | + | 51 | M | N.I. | 327 | 80 | 9 |
| 82 | + | 33 | F | N.I. | 317 | U.D. | 13 |
| 83 | + | 60 | F | HAD | 300 | U.D. | 19 |
| 84 | + | 62 | M | MND | 280 | U.D. | 22 |
| 85 | + | 65 | M | N.I. | 880 | 34 | 27 |
| 86 | + | 58 | F | N.I. | 45 | 692 | 31 |
| 87 | + | 63 | M | N.I. | 951 | <20 | 28 |
| 88 | + | 73 | M | MND | 253 | <20 | 22 |
| 89 | + | 70 | M | N.I. | 422 | <20 | 24 |
| 90 | + | 63 | M | N.I. | 891 | U.D. | 15 |
| 91 | + | 60 | F | N.I. | 264 | U.D. | 14 |
| 92 | + | 61 | F | MND | 1126 | U.D. | 29 |
| 93 | + | 71 | M | N.I. | 892 | U.D. | 18 |
| 94 | + | 74 | M | N.I. | 680 | U.D. | 23 |
| 95 | + | 66 | F | HAD | 681 | 24 | 19 |
| 96 | + | 51 | M | NPI-O | 1053 | 82 | 17 |
| 97 | + | 49 | F | HAD | 518 | 2776 | 14 |
| 98 | + | 67 | F | NPI-O | 480 | 5493 | 24 |
| 99 | + | 43 | F | HAD | 83 | 19531 | 14 |
| 100 | + | 48 | M | NPI-O | 51 | 57880 | 17 |
| 101 | + | 50 | F | HAD | 451 | 20 | 20 |
| 102 | + | 42 | F | HAD | 1422 | U.D. | 22 |
| 103 | + | 62 | F | N.I. | 376 | U.D. | 21 |
| 104 | + | 50 | M | MND | 1254 | U.D. | 14 |
| 105 | + | 49 | F | MND | 761 | 25 | 23 |
| 106 | + | 61 | F | MND | 488 | 34 | 24 |
| 107 | + | 50 | M | NPI-O | 271 | 134 | 11 |
| 108 | + | 55 | F | N.I. | 415 | 20 | 27 |
| 109 | + | 70 | M | N.I. | 544 | U.D. | 26 |
| 110 | + | 61 | M | MND | 353 | U.D. | 27 |
| 111 | + | 48 | F | NPI-O | 731 | U.D. | 3 |
| 112 | + | 54 | F | HAD | 197 | U.D. | 29 |
| 113 | + | 55 | F | MND | 574 | U.D. | 24 |
| 114 | + | 49 | F | NPI-O | 823 | U.D. | 3 |
| 115 | + | 65 | F | MND | 604 | U.D. | 12 |
| 116 | + | 48 | F | N.D. | N.D. | N.A. | N.A. |
| 117 | − | 37 | F | N.D. | N.D. | N.A. | N.A. |
| 118 | − | 45 | M | N.D. | N.D. | N.A. | N.A. |
| 119 | − | 47 | F | N.D. | N.D. | N.D. | N.D. |
| 120 | − | 51 | M | N.D. | N.D. | N.D. | N.D. |
| 121 | − | 68 | M | N.D. | N.D. | N.D. | N.D. |
| 122 | − | 38 | M | N.D. | N.D. | N.A. | N.A. |
| 123 | − | 38 | M | N.D. | N.D. | N.A. | N.A. |
| 124 | − | 42 | F | N.D. | N.D. | N.A. | N.A. |
| 125 | − | 44 | F | N.D. | N.D. | N.A. | N.A. |
| 126 | − | 49 | M | NPI-O | N.D. | N.A. | N.A. |
| 127 | − | 51 | F | N.D. | N.D. | N.A. | N.A. |
| 128 | − | 58 | F | N.D. | N.D. | N.A. | N.A. |
| 129 | − | 61 | F | N.D. | N.D. | N.A. | N.A. |
| 130 | − | 67 | M | N.D. | N.D. | N.A. | N.A. |
| 131 | − | 27 | F | NPI-O | N.D. | N.A. | N.A. |
| 132 | − | 26 | F | N.D. | N.D. | N.A. | N.A. |
| 133 | − | 31 | M | N.D. | N.D. | N.A. | N.A. |
| 134 | − | 34 | M | N.D. | N.D. | N.A. | N.A. |
| 135 | − | 39 | M | N.D. | N.D. | N.A. | N.A. |
| 136 | − | 45 | F | NPI-O | N.D. | N.A. | N.A. |
| 137 | − | 62 | F | N.D. | N.D. | N.A. | N.A. |
| 138 | − | 53 | M | N.D. | N.D. | N.A. | N.A. |
| 139 | − | 39 | F | N.D. | N.D. | N.A. | N.A. |
| 140 | − | 39 | M | N.D. | N.D. | N.A. | N.A. |
| 141 | − | 40 | M | N.D. | N.D. | N.A. | N.A. |
| 142 | − | 47 | M | N.D. | N.D. | N.A. | N.A. |
| 143 | − | 49 | F | N.D. | N.D. | N.A. | N.A. |
| 144 | − | 52 | M | N.D. | N.D. | N.A. | N.A. |
| 145 | − | 58 | M | N.D. | N.D. | N.A. | N.A. |
| 146 | − | 61 | M | N.D. | N.D. | N.A. | N.A. |
| 147 | − | 62 | M | N.D. | N.D. | N.A. | N.A. |
| 148 | − | 33 | M | N.D. | N.D. | N.A. | N.A. |
| 149 | − | 39 | F | N.D. | N.D. | N.A. | N.A. |
| 150 | − | 39 | M | NPI-O | N.D. | N.A. | N.A. |
| 151 | − | 40 | M | N.D. | N.D. | N.A. | N.A. |
| 152 | − | 35 | M | N.D. | N.D. | N.A. | N.A. |
| 153 | − | 39 | F | N.D. | N.D. | N.A. | N.A. |
| 154 | − | 42 | F | N.D. | N.D. | N.A. | N.A. |
| 155 | − | 35 | M | N.D. | N.D. | N.A. | N.A. |
| 156 | − | 52 | M | N.D. | N.D. | N.A. | N.A. |
| 157 | − | 55 | M | N.D. | N.D. | N.A. | N.A. |
| 158 | − | 43 | M | N.D. | N.D. | N.A. | N.A. |
| 159 | − | 45 | F | N.D. | N.D. | N.A. | N.A. |
| 160 | − | 30 | F | N.D. | N.D. | N.A. | N.A. |
| 161 | − | 31 | M | N.D. | N.D. | N.A. | N.A. |
| 162 | − | 42 | M | NPI-O | N.D. | N.A. | N.A. |
| 163 | − | 39 | M | N.D. | N.D. | N.A. | N.A. |
| 164 | − | 43 | M | N.D. | N.D. | N.A. | N.A. |
| 165 | − | 45 | M | N.D. | N.D. | N.A. | N.A. |
| 166 | − | 38 | M | N.D. | N.D. | N.A. | N.A. |
| 167 | − | 39 | M | N.D. | N.D. | N.A. | N.A. |
| 168 | − | 38 | M | N.D. | N.D. | N.A. | N.A. |
| 169 | − | 43 | M | N.D. | N.D. | N.A. | N.A. |
| 170 | − | 43 | F | N.D. | N.D. | N.A. | N.A. |
| 171 | − | 47 | F | N.D. | N.D. | N.A. | N.A. |
| 172 | − | 52 | F | N.D. | N.D. | N.A. | N.A. |
| 173 | − | 56 | M | N.D. | N.D. | N.A. | N.A. |
| 174 | − | 55 | F | N.D. | N.D. | N.A. | N.A. |
| 175 | − | 57 | M | N.D. | N.D. | N.A. | N.A. |

Notes:
M: male; F: Female; N.A.: not applicable; N.D.: not determined; N.R.: not recorded. NI: No impairment; MND: Mild Neurocognitive Disorder; HAD: HIV associated dementia; NPI-O: Neurocognitive impairment other or UD: undetectable.

Isolation of human PBMCs and CD4⁺ T lymphocytes. After removing the plasma, PBMCs were isolated by over-layering with Ficoll-Paque (Cat #GE17-1440-02, Amersham Bioscience, Uppsala, Sweden) according to the procedure described by the manufacturer. PBMCs were isolated within 4 h of blood draw. All described analysis was performed on freshly isolated blood.

Dye uptake and time-lapse microscopy. To characterize the functional state of Panx-1 channels, dye-uptake experiments using ethidium (Etd) bromide were performed (Cat #15585011, ThermoFisher, Grand Island, N.Y., USA). Cells were washed twice in HBSS and then exposed to Locke's solution (containing 154 mM NaCl, 5.4 mM KCl, 2.3 mM CaCl₂, 5 mM HEPES, and pH 7.4) with 5 μM Etd and time-lapse microscopy were then performed. Phase-contrast and fluorescence microscopy with time-lapse imaging were used to record cell appearance and fluorescence-intensity changes in each condition. Fluorescence was recorded every 30 s. The NIH ImageJ program was used for off-line image analysis and fluorescence quantification. For data representation and calculation of Etd uptake slopes, the average of two independent background fluorescence (FB) (expressed as A.U.) was subtracted from mean fluorescent intensity (F1). Results of this calculation (F1-FB), for at least 20 cells, were averaged and plotted against time (expressed in minutes). Slopes were calculated using Microsoft Excel software and expressed as A.U./min. The microscope and camera settings remained the same in all experiments. Dead cells or cells with a damaged plasma membrane were identified during the time-lapse microscopy as a result of their nonspecific Etd uptake rate, determined by lack of time dependency and stability in dye uptake (not inhibited by channel blockers), and were not quantified.

ATP Assay. Plasma/serum was collected before PBMC separation, and ATP concentration was determined using the ATPlite luminescence assay system (PerkinElmer, Mass.) by combining 100 μL of the sample with 100 μL of ATPlite reagent. Luminescence was measured using a PerkinElmer EnVision Multilabel Plate Reader. The extracellular concentration of ATP was determined by comparing sample luminescence to a standard curve generated using ATP standards provided by the manufacturer. To assure rigor in these determinations, some samples were submitted for blinded analysis of ATP levels using mass spectrometry (University of North Dakota, N. Dak.).

Analysis of IL-1β and PGE₂ release. Plasma/serum was collected, divided into aliquots, and stored at −80° C. There were no freeze-thaw cycles before analysis. Plasma/serum was analyzed for TNF-α, IL-1β (Quantikine ELISA kit; R and D Systems, Minneapolis Minn., USA) and PGE₂ (Abcam, Cambridge, Mass., USA) by enzyme-linked immunosorbent assay (ELISA) according to the manufacturer's instructions.

Blood-brain barrier (BBB) model: This in vitro BBB model consists of primary human BMVEC and primary human astrocytes in co-culture on opposite sides of a gelatin-coated, 3 μm pore-size tissue culture insert (Falcon, B D, Franklin Lakes, N.J.) as described by the inventors [37-41]. Co-cultures were maintained for three days to enable contact between astrocyte endfeet with BMVEC on the opposite side of the model as described [37]. After this, the BBB model was treated with different ATP concentrations (Cat #A1852, Sigma Chemical Co., St. Louis, Mo., USA), and BBB permeability was measured using BSA conjugated to Evans Blue, as described by the inventors [37].

Transmigration assays of mononuclear cells across the model of the human BBB. Three x 105 PBMCs in M199 culture medium (Cat #31100035, ThermoFisher, Grand Island, N.Y., USA) with 10% FBS was added to the top of each tissue culture insert as described [37, 42]. After 24 h the number of cells that had transmigrated in response to CCL2 (100 or 500 ng/ml) or without chemoattractant added to the lower chamber was analyzed by FACScan using premixed human CD45 (RRID: AB_10852703) and CD14 (RRID: AB_10598367) monoclonal antibodies conjugated to FITC and PE, respectively [37, 42].

Statistical analysis. Statistical analyses were performed using Prism 5.0 software (GraphPad Software, Inc., San Diego, Calif.). Analysis of variance was used to compare the different groups; *P≤0.001 for all statistical analyses performed in this study.

Results.

Participant Demographics. 175 plasma/serum samples were collected from uninfected (n=60) and HIV-infected individuals (n=115). There were no differences between the HIV-negative and HIV-positive individuals in age (HIV-positive, mean=47.9±12.2 years; HIV-negative, mean=45.3±10.1 years; Table 1 and 2) and gender (HIV-positive=39% female and 61% male; HIV-negative=39% female and 61% male; Table 1 and 2). The HIV-positive cohort had an average of 15.9±6.8 years of living with HIV, an average CD4 count of 323.2±285.7 cells/μl, and mean plasma HIV RNA of 14,452±41,779 log copies/ml (range from undetectable to 57,880). Approximately 28% of participants had an undetectable viral load (see Table 1 and 2). Among the HIV-positive participants, 69% had some degree of cognitive impairment, as determined by neuropsychological testing (Table 5).

To assure an unbiased assessment of the serum/plasma samples, all samples were received and analyzed blindly. Only after all the data was acquired, clinical and HIV status was requested. With regard to HIV associated CNS disease, the population ranged from no impairment to HIV-associated dementia. Table 5 summarizes the information of each individual analyzed including age, gender, years with HIV, CD4 count, plasma HIV RNA copies, and cognitive status.

TABLE 5
Demographic of HIV positive and HIV negative participants

| | HIV Positive | HIV Negative |
|---|---|---|
| | (n = 115) | (n = 60) |
| | Mean ± SD | Mean ± SD |
| Patient Demographics | | |
| Age (Years) | 47.89 ± 12.23 | 45.3 ± 10.12 |
| % Female | 39% | 39% |
| % Male | 61% | 61% |
| Years w/HIV | 15.87 ± 6.8 | N/A |
| Immunovirologic information | | |
| CD4 T cell count, cells/μl | 323.2 ± 285.7 | N/A |
| Plasma HIV RNA, log copies/ml | 14,452 ± 41,779 | N/A |
| % w/undetectable viral loads | 47% | N/A |
| | 28% | N/A |
| % w/cognitive impairment | 69% | N/A |

N/A = Not Applicable

Pannexin-1 channels are in a closed state in uninfected individuals. The inventors' previous published data indicate that binding of HIV to CD4 and CCR5/CXCR4 co-receptors results in the opening of Panx-1 channels, thus enabling the virus to fuse with the plasma membrane. In contrast, in uninfected samples, the channel remains closed [29, 33, 43]. However, the chronic effects of HIV infection on Panx-1 channel activity were unknown. To determine the status of the channel, Panx-1 channel opening was determined by Ethidium (Etd, 5 μM) uptake rate Ethidium only crosses the plasma membrane in healthy cells by passing through specific large channels, such as Connexin (Cx) and Pannexin (Panx) hemichannels, and its rate of intracellular accumulation is reflective of channel opening [44, 45].

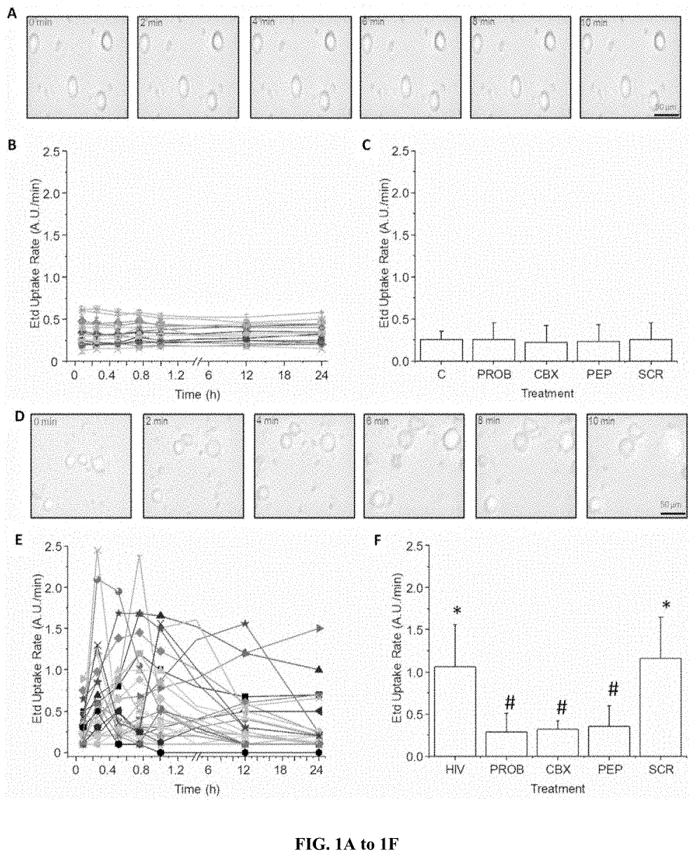

FIGS. 1A to 1F show: PBMCs isolated from uninfected individuals maintain the pannexin-1 channels in a closed stage. In contrast, PBMCs isolated from HIV-infected individuals have a spontaneous opening of pannexin-1 channels. (FIG. 1A) Uptake of Ethidium (Etd) was quantified by time-lapse microscopy at different time points up to 24 h, the last point assayed. Ethidium can only cross the plasma membrane through hemichannels due to its high molecular weight. Only live cells with increasing uptake of Etd were quantified to discount Etd uptake of dead cells. A representative image of live cell imaging for 10 min of recording. (FIG. 1B) Quantification of Etd uptake rate indicates that in all individuals analyzed, the hemichannels on the surface of PBMCs were in a closed stage. Each line represents data from a single individual. (FIG. 1C) Blockers of pannexin-1 channels such as Probenecid (PROB, 50 μM), carbenoxolone (CBX, 50 μM), pannexin-1 blocking peptide (PEP, 300 μM). Scrambled peptide (SCR) did not show any unspecific effect. (n=30). The data are expressed as mean±SD. (FIG. 1D) As indicated in FIG. 1, uptake of Ethidium (Etd) was quantified by time-lapse microscopy at different time points up to 24 h, the last point assayed. A representative image of live cell imaging for 10 min of recording. Fluorescence is accumulated in a time-dependent manner inside of PBMCs. Most of these PBMCs come from HIV-infected cells with and without peripheral viral replication (see table 1). (FIG. 1E) Quantification of Etd uptake rate indicates that hemichannels on the surface of PBMCs were in an open stage in all individuals analyzed (n=60). Each line represents data from a single individual. (FIG. 1F) Blockers of pannexin-1 channels such as Probenecid (PROB, 50 μM), carbenoxolone (CBX, 50 μM), pannexin-1 blocking peptide (PEP, 300 μM) were able to prevent the opening of the pannexin-1 channel and Etd uptake observed in HIV-infected conditions. Scrambled peptide (SCR) did not show any unspecific effect. (n=60). * Correspond to significance compared to control conditions as observed in FIG. 1, *p≤0.005, n=60; #p≤0.003, n=60, as compared to HIV conditions. The data are expressed as mean±SD.

No significant changes in Etd uptake rate were detected in PBMCs obtained from uninfected individuals (FIGS. 1A and 1B, n=45 different individuals). The inventors performed experiments with up to 25 h of recording with minimal to no Etd uptake detected (FIG. 1B). Pooling all the data from PBMCs obtained from 45 different uninfected individuals indicated a low to undetectable dye uptake (FIG. 1C, control). Incubation with the Panx-1 blockers probenecid (Prob, 500 μM) (Cat #P8761), carbenoxolone (CBX, 500 μM) (Cat #C4790, Sigma Chemical Co. St. Louis, Mo., USA), and ¹⁰Panx peptide (200 μM, PEP) (Cat #3348, Tocris, Minneapolis, Minn., USA) [46, 47] did not affect basal Etd uptake observed in PBMCs obtained from uninfected individuals (FIGS. 1A and 1B). Further, connexin 43 (Cx43) hemichannel blockers such as lanthanum (La³⁺) (Cat #449830, Sigma Chemical Co. St. Louis, Mo., USA), a general Cx hemichannel blocker, or Cx43^{E2}, a peptide that specifically blocks Cx43 hemichannels [44, 48, 49] did not have any effect on the low dye uptake observed in uninfected PBMCs (FIG. 1C). No toxic or nonspecific effects of these blockers alone were detected (data not shown). Furthermore, as a control, exposure of PBMCs obtained from uninfected individuals to HIV_{ADA} (20 ng/ml and 0.001 MOI) resulted in a fast and transient opening of the channel as described by the inventors [33].

Pannexin-1 channels are constitutively open on PBMCs isolated from HIV-infected individuals. To assess the opening of the Panx-1 channels on PBMCs isolated from HIV-infected individuals, the inventors used a similar approach as described above. The inventors analyzed Panx-1 channel opening using dye uptake rate on PBMCs isolated from 60 different HIV-infected individuals, mostly with low to undetectable replication, as described in Table 1. All samples analyzed had significant dye uptake, even though most of the cells did not have the circulated virus or had undetectable concentrations of the extracellular virus during the recording (FIG. 1D, IE, and data not shown). The inventors' previous data indicated that most openings of Panx-1 channels were transient and induced by acute exposure to virus [29, 33, 43]. However, the data herein shows that chronic HIV infection had profound effects on Panx-1 channel opening via an unknown mechanism that is independent of CD4, CXCR4 or CCR5 engagement for the virus, because soluble CD4, TAK779 or AMD3100 (Cat #SML0911, Cat #A5602, Sigma Chemical Co., St. Louis, Mo., USA) did not prevent the spontaneous Panx-1 channel opening (data not shown). As indicated in FIG. 1E, each HIV-infected individual, had a different time course of Panx-1 channel opening (FIGS. 1D, E, and F). However, all HIV-infected individuals analyzed had spontaneous Panx-1 channel opening. The opening of the Panx-1 channels was independent of the age of the individual, gender, years with HIV, CD4 count, HIV replication, ART, and cognitive status.

Pannexin-1 channel opening on PBMCs isolated from chronic HIV-infected individuals was sensitive to probenecid (Prob, 500 μM), carbenoxolone (CBX), and ¹⁰Panx peptide (200 μM, PEP)—all Panx-1 blockers (FIG. 1F)—indicating that dye uptake was mediated by Panx-1 channels. In contrast, Lanthanum (La³), a general Cx hemichannel blocker, or Cx43^{F}, an peptide that blocks Cx43 hemichannels [44, 48, 49], did not affect the Etd uptake observed in the PBMCs isolated from the HIV-infected population (data not shown), suggesting that Cx43 hemichannels were not open during these studies. Therefore, a constitutive opening of Panx-1 channels could explain the chronic inflammation observed in the HIV-infected population, even in the current ART era.

Serum/plasma obtained from HIV-infected individuals had elevated concentrations of inflammatory molecules released upon the opening of pannexin-1 channels.

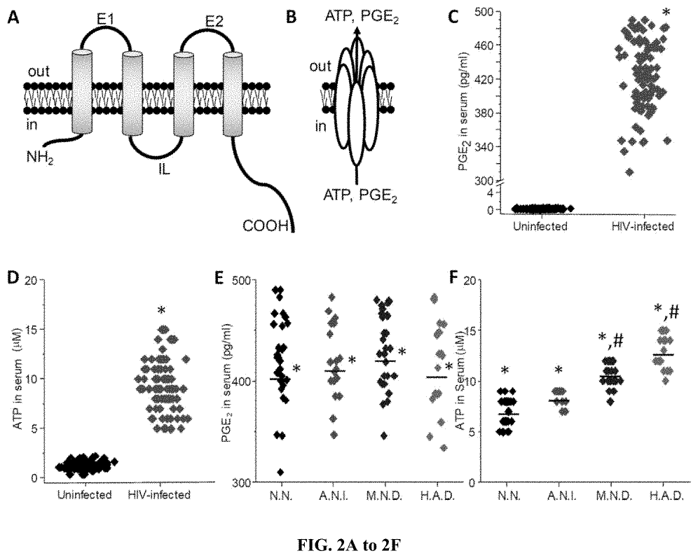

FIGS. 2A to 2F show: Opening of Pannexin-1 channels on PBMCs is associated with increased circulating levels of PGE₂ and ATP in the plasma/serum of HIV-infected individuals. (FIG. 2A) Representation of a pannexin-1 protein and the structure of the pannexin-1 channel. (FIG. 2B). Opening of Pannexin-1 channels on the surface of the cells enables the release of PGE₂ and ATP. (FIG. 2C) Quantification of PGE₂ in serum/plasma by ELISA. Uninfected individuals had no significant levels of PGE₂ detected (black signs). In contrast, serum/plasma obtained from HIV-infected individuals described in Table 1, indicates that all HIV-infected individuals have increased levels of circulating PGE₂ despite good CD4 counts and low to undetectable viral replication (red signs). (FIG. 2D) Quantification of ATP circulating levels in serum/plasma using ATP light. ATP levels in uninfected individuals are low and unstable in solution (n=60). However, in all samples (serum/plasma) from HIV-infected individuals, ATP levels were high (n=115, *p≤0 005 as compared to uninfected conditions). (FIG. 2E) If the PGE₂ data presented in C were breakdown into cognitive status, N.N.: Neurocognition normal; A.N.I.: Asymptomatic Neurocognitive Impairment: M.N.D.: Mild Neurocognitive Disorder; and H.A.D: HIV-Associated Dementia, no significant differences were observed (n=115, *p≤0.005 as compared to control uninfected conditions). (FIG. 2F) If the ATP circulating levels presented in D were breakdown into cognitive impairment was an association between M.N.D. and H.A.D.. circulating ATP levels can be used as a biomarker of cognitive disease in the HIV-infected population (n=115, *p≤0.005 as compared to control uninfected conditions and #p≤0.002 as compared to N.N. and A.N.I.). Thus, changes in cognition or CNS compromise are associated with increased levels of ATP. The data are expressed as mean±SD.

The inventors had previously shown that Panx-1 channel opening in response to HIV binding to CD4 and CCR5 or CXCR4 enables ATP to be released and subsequently activates purinergic receptors, thereby allowing entry of the virus into uninfected macrophages [4]. ATP is released into the extracellular space via three main mechanisms; neuronal secretion (vesicular release), cell death (plasma membrane compromise), and the opening of Panx-1 channels [50-54](FIGS. 2A and B). Thus, to determine whether the constitutive opening of Panx-1 channels on circulating PBMCs obtained from HIV-infected individuals is also associated with higher levels of intracellular mediators released through the channel, the inventors determined circulating levels of PGE₂, ATP, TNF-α and IL-1p in serum/plasma of uninfected and HIV-infected individuals.

As expected, low to undetectable levels of PGE₂ and ATP were found in the uninfected population (FIGS. 2C and D, uninfected, respectively, n=60). No detectable levels of TNF-α or IL1β were detected in the serum of uninfected individuals (data not represented). In contrast, high circulating levels of PGE₂ and ATP were detected in all serum/plasma samples analyzed from HIV-infected individuals (FIGS. 2 C and D, HIV-infected, respectively, n=115). The differences in circulating levels of PGE₂ and ATP were independent of the age of the individual, gender, years with HIV, CD4 count, and HIV replication (data not represented). In addition, no detection of TNF-α or IL-1β was found in any condition analyzed (data not shown). In conclusion, the inventors demonstrated that intracellular factors such as PGE₂ and ATP, both released through the opening of Panx-1 channels, are constitutively released into the circulation of all HIV-infected individuals.

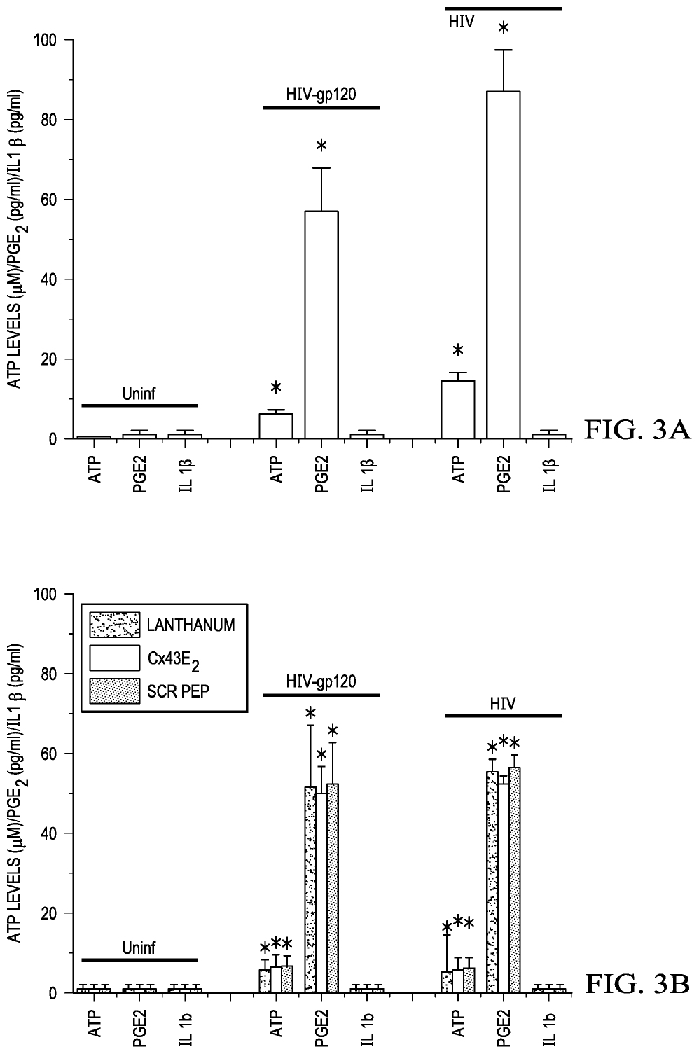

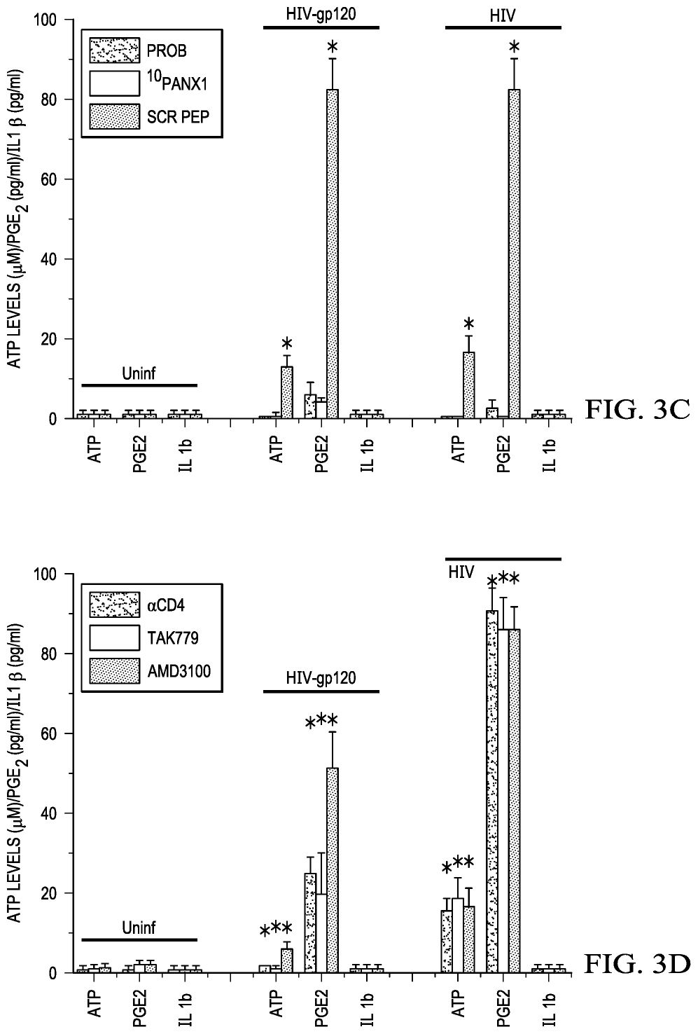

FIGS. 3A to 3D show: PBMCs isolated from HIV-infected individuals release ATP and PGE₂ in a pannexin-1 dependent, but not Cx43 hemichannel, manner. (FIG. 3A) PBMCs were isolated from uninfected and HIV-infected individuals with and without cognitive impairment, washed, and determinations of ATP, PGE₂, and IL 1p were performed after 15 and 30 min post-stimulation. Uninfected cells (Uninf) PBMCs did not release significant amounts of ATP, PGE₂, and IL1β. However, upon treatment with the recombinant protein, HIV-gp120 (HIV-Bal), pannexin-1 channels become open, and a significant amount of ATP and PGE₂ were released. PBMCs obtained from HIV-infected individuals (HIV), without further stimulation, release ATP, and PGE₂ into the medium. Thus, pannexin-1 channels are open, as described in FIG. 2. In both types of PBMCs, uninfected, and HIV, no detection of IL1β was found. (FIG. 3B) Blocking the opening of Cx43 hemichannels on the surface of PBMCs using lanthanum, or the extracellular peptide (Cx43E₂) did not alter the release of ATP and PGE₂. Scrambled peptide (Scr pep) did not alter the secretion of ATP and PGE₂. (FIG. 3C) Blocking the opening of pannexin-1 channels on the surface of PBMCs prevented the secretion of ATP and PGE₂ in uninfected cells treated with HIV-gp120 (HIV-gp120) or in PBMCs obtained from HIV-infected individuals (HIV). (FIG. 3D) The application of soluble CD4 to compete with the binding of HIV-gp120 or the virus prevented secretion of ATP and PGE₂ in response to HIV-gp120. However, in PBMCs isolated from HIV-infected individuals, no significant effects of sCD4, CCR5 (TAK779) or CXCR4 (AMD3100) blockers were observed. (n=6, *p≤0.003 as compared to control uninfected conditions). The data are expressed as mean±SD.

Circulating levels of ATP are predictive of cognitive impairment in the HIV-infected population. As described above and by others, under physiological conditions, low concentrations of circulating ATP are found (1-2 μM) [55, 56]. However, the inventors found that all HIV-positive individuals analyzed contained significantly higher levels of circulating PGE₂ and ATP in their plasma/serum relative to uninfected individuals, as described above (*p≤0.005; FIGS. 2C and D). PGE₂ did not associate with cognitive impairment (FIG. 3E). However, when circulating ATP levels were stratified according to cognitive impairment, the inventors found that individuals with no neurocognitive impairments (N.N.) had significantly lower plasma ATP than those with asymptomatic neurocognitive impairment (A.N.I.), mild neurocognitive disorders (M.N.D.), or HIV-associated dementia (H.A.D.) (*p≤0.005; FIG. 2F, n=60 for uninfected and n=115 for HIV-infected individuals). The inventors detected that concentrations higher than 8 μM were associated with cognitive disease (M.C.M.D. or H.A.D.) (FIG. 2F). Thus, circulating levels of ATP could be used as a biomarker of cognitive impairments in the HIV-infected population.

PBMCs isolated from HIV-infected individuals release ATP and PGE₂, even without stimulation. To determine whether the opening of Panx-1 channels contributed to the extracellular levels of PGE₂ and ATP, the inventors determined the acute release of these factors using PBMCs obtained from uninfected and HIV-infected individuals. Uninfected and HIV-infected PBMCs were isolated as described above. PBMCs isolated from uninfected individuals did not show the opening of Panx-1 channels (see FIG. 1B), and no significant release of PGE₂, ATP, and IL-1p into the medium was detected after 15 and 30 min in culture (FIG. 3A, uninf). However, gp120 treatment (1 μg/ml, from HIV_{B}:a) of uninfected PBMCs induced opening of the Panx-1 channel and resulted in the release of ATP and PGE₂ (5.9 f 1.3 μM and 56.8 f 11.1 pg/ml, respectively), but not IL1β, after 30 min (FIG. 3A, HIV-gp120). In contrast, PBMCs isolated from HIV-infected individuals washed and placed in culture resulted in ATP and PGE₂ release into the medium even without any stimulation (14.1±2.4 μM and 87.0±10.1 pg/ml, respectively, FIG. 3A, HIV).

The release of ATP and PGE₂ in response to HIV-gp120 in PBMCs obtained from uninfected individuals or the spontaneous release observed in PBMCs obtained from HIV-infected individuals was not dependent on the opening of Cx-43 hemichannels. Lanthanum, a general Cx hemichannel blocker or an extracellular blocking peptide to the extracellular loop 2 (FIG. 3B, Cx43E₂) did not affect the release of ATP or PGE₂ in response to HIV-gp120 using uninfected cells or the release of ATP or PGE₂ in PBMCs from HIV-infected individuals (FIG. 3B).

In contrast, the release of ATP and PGE₂ in response to HIV-gp120 in PBMCs obtained from uninfected individuals or the spontaneous release observed in PBMCs obtained from HIV-infected individuals were sensitive to Panx-1 channel blockers (FIG. 3C). Probenecid (Prob, 500 μM) or ¹⁰Panx1 peptide (300 μM) treatment prevented the release of ATP and PGE₂ in PBMCs isolated from uninfected and HIV-infected individuals (FIG. 3C). No effects on PGE₂ and ATP were detected using the scrambled Panx-1 peptide (Scr pep, FIG. 3C) (Cat #3708, Tocris, Minneapolis, Minn., USA).

Furthermore, blocking gp120 binding to CD4 (soluble CD4 protein, Cat. 4615, NIH repository), CCR5 (TAK779, 5 μM) or CXCR4 (AMD3100, 5 μM) also prevented the release of ATP and PGE₂ in response to gp120 treatment of PBMCs from uninfected individuals (FIG. 3D, HIV-gp120). However, none of the blockers for CD4, CCR5, or CXCR4 reduced the spontaneous opening observed in PBMCs obtained from HIV-infected individuals (FIG. 3D).

Thus, in uninfected PBMCs, HIV stimulation is required to induce the opening of Panx-1 channels as well as to release ATP and PGE₂. Treatment of uninfected PBMCs with TNF-α (10 ng/ml) (Cat #11371843001), IL-1p (10 U/ml) (Cat #SRP3083), IFN-γ (10 ng/ml) (Cat #SRP3058) or LPS (1 μg/ml) (Cat #L2630, Sigma Chemical Co., St. Louis, Mo., USA) for 30 min does not induce opening of the pannexin-1 channels or secretion of PGE₂ and ATP. Thus, the secretion of these factors was not associated with immune activation, rather with HIV infection. In contrast, the release of ATP and PGE₂ from PBMCs isolated from HIV-infected cells was independent of CD4 and chemokine receptor stimulation (FIG. 3D). Thus, the mechanism of Panx-1 opening and ATP and PGE₂ release is not dependent on viral or gp120 binding to host receptors in the HIV infected population.

Thus, chronic HIV-infected PBMCs may be a major contributor to circulating levels of PGE₂. and ATP observed in the serum of HIV-infected patients.

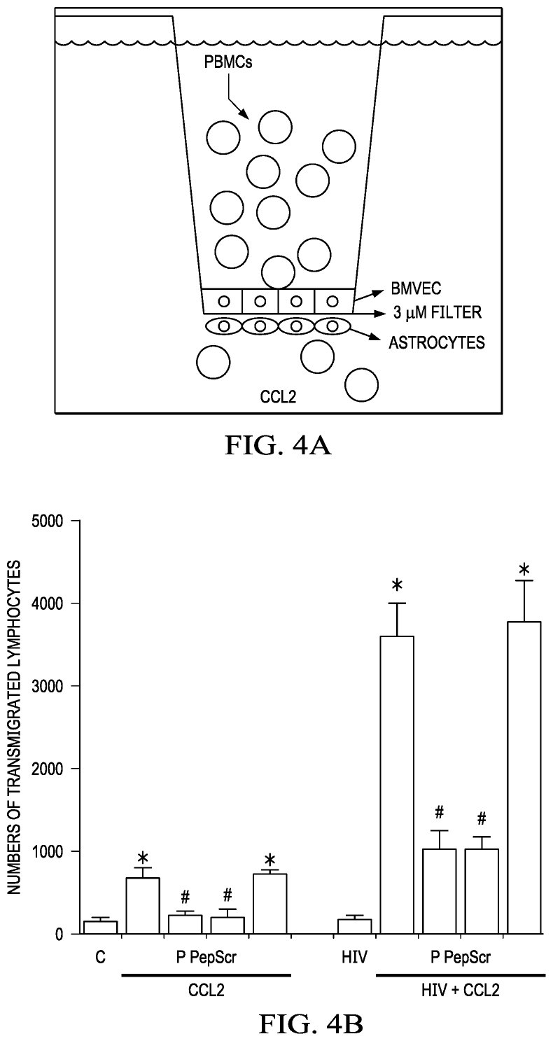

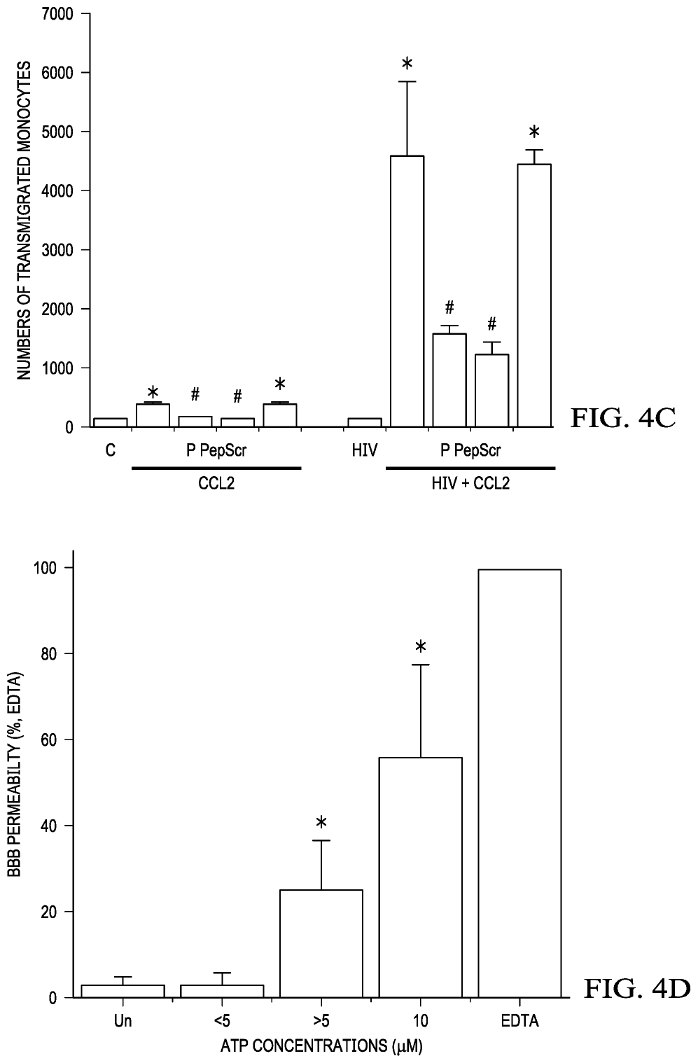

FIGS. 4A to 4D show: Transmigration uninfected and HIV-infected PBMCs (HIV_{ADA}) is pannexin-1 dependent, and secreted ATP contributes to BBB disruption. (FIG. 4A) A schematic representation of the EC/astrocyte blood-brain barrier (BBB) co-culture model. After 24 h post-transmigration cells in the bottom chamber were collected and stained for CD14 and monocytes and CD3 for lymphocytes. (FIG. 4B and FIG. 4C) Uninfected (C) or HIV-infected human PBMCs were added to the top chamber of the BBB model, consisting of co-cultured human ECs and astrocytes in the absence or presence of CCL2 (100 ng/ml) in the bottom chamber. In addition, pre-incubation of PBMCs with probenecid (P) or pannexin-1 peptide (Pep) blockers reduced the transmigration of uninfected and HIV-infected lymphocytes and monocytes.

Scrambled peptide (Scr) did no altered transmigration of lymphocytes or monocytes in response to CCL2. *p S 0.05 as compared to control conditions without CCL2. #p≤0.003 as compared to CCL2 conditions. (FIG. 4D) The permeability of the BBB, without PBMCs, only with added ATP into the top chamber, was determined using albumin conjugated to Evans blue. Untreated BBB model (Un) and low levels of ATP detected in uninfected individuals (5 μM) did not result in changes in BBB permeability. ATP concentrations higher than 5 μM (7 and 9 μM) increased the permeability of the barrier. ATP concentrations (10 μM) detected in HIV-infected individuals compromised BBB permeability. Control permeability by treating the BBB model with EDTA was used. *p≤0.005 as compared to untreated conditions. The data are expressed as mean±SD of 6 experiments.

Opening of pannexin-1 channels is required for enhanced transmigration of HIV-infected lymphocytes and monocytes in response to CCL2. To determine transmigration, the inventors used a blood-brain barrier (BBB) model that selects mostly HIV-infected PBMCs to transmigrate into the brain side in response to CCL2 [37, 42, 57-60] (FIG. 4A).

To determine whether Panx-1 channels, as well as extracellular levels of ATP compromise, participate in the transmigration of HIV-infected cells across the BBB in response to CCL2 (100 ng/ml) (Cat #279-MC, R&D Systems, Minneapolis, Md., USA), the inventors determined transmigration in the presence and absence of Panx-1 channel blockers, probenecid (P) or ¹⁰Panx-1 blocking peptide (Pep). The addition of uninfected PBMCs to the top chamber of the BBB model in the absence of CCL2 did not affect the baseline permeability of co-cultures (data not shown) and minimally affected transmigration of lymphocytes and monocytes across the BBB (FIGS. 4B and C, for lymphocytes and monocytes, respectively). Preincubation of the PBMCs with Probenecid (P) or ¹⁰Panx-1 blocking peptide (Pep) and subsequent transmigration induced by CCL2 reduced transmigration to control levels (C, FIGS. 4 B and C).

PBMCs infected with the HIV_{ADA} were added to the top chamber of BBB co-cultures without CCL2 in the bottom chamber (HIV). After 24 h, there was neither significant transmigration, as described above (FIGS. 4B and C, HIV), nor disruption of BBB impermeability under these conditions (data not shown). The addition of CCL2 to the lower chamber induced high levels of HIV-infected cell transmigration as described above (FIGS. 4B and C) and resulted in very significant increases in BBB permeability (data not shown), as compared to co-cultures after uninfected PBMC transmigration (FIGS. 4B and C, bar labeled C on the X-axis). The preincubation of HIV_{ADA} infected PBMCs with Probenecid (P), or ¹⁰Panx-1 blocking peptide (Pep) prevented transmigration induced by CCL2 and reduced BBB disruption (FIGS. 4B and C). Furthermore, the addition of oATP (100 μM, a purinergic receptor blocker) (Cat #A6779) or apyrase (100 units/ml, an enzyme that catalyzes the hydrolysis of ATP) (Cat #A6535, Sigma Chemical Co., St. Louis, Mo., USA) to the co-culture blocked transmigration as well as BBB disruption, supporting the hypothesis that opening of Panx-1 channels results in the local ATP release and subsequent activation of purinergic receptors (data not shown).

High circulating levels of ATP present in the circulation of HIV-infected individuals compromise BBB function. To determine the role of circulating ATP in the HIV-infected population, the inventors measured BBB permeability and leukocyte transmigration across an in vitro human BBB model. Both aspects are observed in HIV-infected individuals and several animal models of HIV-brain compromise [60-62]. In FIG. 4A is a representation of the BBB model used to examine permeability and transmigration. The inventors previous published data indicated that HIV infection plus CCL2 correspond to a unique combination that favors BBB disruption and enhanced transmigration of leukocytes into the CNS [42, 60, 63]. However, the mechanism mediating this specific effect of HIV infection and BBB disruption was unknown.

The addition of ATP to the luminal side of the model (blood side) to concentrations lower than 2 μM minimally affected BBB permeability (FIG. 4D, Un). Increasing concentrations of ATP similar to the ones observed in the HIV-infected population (higher than 5-50 μM, FIG. 4D), strongly compromised BBB permeability even in the absence of an HIV component as detected. As a positive control, EDTA (Cat #E6758, Sigma Chemical Co., St. Louis, Mo., USA) was used to disrupt the barrier (FIG. 4D, EDTA).

Most HIV-infected individuals, despite effective ART, have chronic systemic and brain inflammation. Currently, a major public health problem is the increased prevalence of mild forms of neurocognitive impairment in 50-60% of HIV-infected individuals [64, 65]. HIV invades the brain early after primary infection, and despite effective ART, HIV remains in sanctuary sites as viral reservoirs [66-68]. Although the extent of HIV infection in the CNS is limited (perivascular macrophages, microglia, and astrocytes), the extent of neuropathogenesis observed does not correlate with viral replication. Thus, it is important to identify biomarkers of CNS disease as well as to understand mechanisms causing CNS compromise better.

Currently, CNS damage is evaluated by common neurophysiological testing batteries and by imaging techniques or determination of novel biomarkers in the CSF [69-72]. However, there are no good systemic biomarkers of CNS disease. Probably, the most promising CSF biomarker of neuronal injury is neurofilament (NFL) levels that were elevated before the onset of dementia, thus providing an early, predictive biomarker of asymptomatic progression, but it is a result of neuronal destruction [73-75]. The normalization of CSF NFL levels is also correlated with the initiation of ART [76-79]. CSF NFL levels are also elevated in several neuroinflammatory and neurogenerative diseases, as well as stroke and other associated conditions. The NFL is thus not predictive of HIV-associated CNS alone, and may just be a late representation of large caliber axon destruction. Furthermore, NFL levels in the periphery are not representative of the damage within the CNS. Also, CSF samples are difficult and painful to obtain. Thus, the necessity for a peripheral CNS biomarker of disease is urgent.

Various additional potential biomarkers of HIV CNS disease have been proposed by several groups, including neopterin, BCL11b, beta-2-microglobulin, several markers of inflammation (sCD163, CCL2, TNF-α, IL-6, sCD14, and CXCL10), and interferon-alpha [80-83]. However, all these biomarkers are associated with already occurring tissue damage and do not predict future damage. Recently, NIH sponsor groups such as CHARTER, NNTC, Neuroimaging Consortium, and proteomic determinations done by several laboratories have indicated that local alterations in metabolites could predict disease onset. Neuroimaging data also provides essential several potential biomarkers of cognitive disease in the HIV infected population such as N-acetylaspartate (NAA) and creatine (Cr) levels in different regions of the brain; however, the results are contradictory and most of the time are region specific [76, 84-86]. Moreover, most of these studies analyzed ratios of these metabolites with respect to Cr, assuming a constant expression of Cr [87-89]. However, Cr expression is variable with age, trauma, and inflammation. Other key metabolites in the brain are glutamate and glutamine that has been associated with cognitive disease in the HIV infected population [86]. The combination of these metabolites has become extremely important due that glutamate and creatine are highly abundant in the brain, but recently has been demonstrated that HIV reservoirs can survive in an alternative source of carbons such as glutamate and glutamine [90]. Additional metabolites and mitochondrial markers are citrate, creatine, glutamine, glucose, inositol, glutamic acid, and CSF mtDNA [82, 91-94]. Thus, HIV infection, even in the absence of replication, has profound effects on the metabolism of infected cells, which may help to perpetuate the virus or promote the survival of infected cells. ATP dysregulation will contribute to the discovery of new biomarkers of CNS disease in the HIV-infected population that will allow for early intervention before CNS damage becomes irreversible.

Recently the inventors identified Panx-1 channels, which are membrane-bound large pore channels ubiquitously expressed in all tissues [95], as a key protein and channel involved in HIV pathogenesis [33]. Normally, these channels are in a closed state. However, the inventors identified that binding of the virus to its receptor (CD4) and co-receptors (CXCR4 and/or CCR5) induces the opening of Panx-1 channels resulting in ATP release through the channel pore and subsequent purinergic receptor activation to allow HIV entry into immune cells. Pannexins are structurally similar to connexins (Cxs), although they share no sequence homology. Pannexins consist of a cytosolic N-terminal domain, four transmembrane domains with two extracellular loops, and a cytosolic C-terminal domain [96, 97]. Currently, there are only a few mechanisms that result in the opening of Panx-1 channels, but most have been described in in vitro conditions [96-103]. It was found that pathogens, including HIV, can “hijack” this channel to accelerate the disease process [29, 31-33]. Thus, the opening of Panx-1 channels is essential for infectivity [33], but any link to NeuroHIV was unknown. These data using patient samples show that chronic HIV infection also results in the unspecific opening of Panx-1 channels and the release of several intracellular inflammatory factors into the extracellular space, including ATP and PGE₂. Normally, secreted ATP is one of the strongest signaling molecules in the development of inflammation. Given the biological potency of ATP, the control of the duration and magnitude of the cellular responses to ATP is crucial [104, 105]. However, its half-life is short and restricted to small areas [106]. Thus, high levels of this nucleotide in all HIV-infected individuals analyzed was a surprise. Most ATP processing is mediated by ecto-ATPase, which converts ATP and ADP into AMP. For example, the ecto-5′-nucleotidase, CD73, can complete the dephosphorylation process and convert the monophosphate into adenosine. This point is critical because ATP is one of the more powerful pro-inflammatory cytokines, whereas adenosine has a potent immunosuppressive effect [107, 108]. These data, which demonstrate that circulating ATP is more stable in the HIV-infected population, also showed a significant decrease in adenosine, showing that all HIV-infected individuals have problems in removing phosphate from complex structures.

The main mechanism of increased levels of ATP in the HIV-infected population is unknown. Normally, extracellular ATP is degraded quickly, but why remain a significant amount in the blood of HIV infected individuals is unknown. Despite that the number of individuals analyzed was significant (n=175), there was not association of the high levels of ATP present in the HIV infected individuals with several comorbidities such as alcohol, inflammation, stroke, or infections as well as genetic factors such as ethnicity or gender. However, an increase number of samples could dissect these points. Furthermore, longitudinal studies also could provide a better time line of ATP dysregulation, BBB disruption, and cognitive compromise. A surprising result was that all HIV infected individuals analyzed have a specific type of inflammation mediated by PGE₂ and ATP, but not represented in changes in the usual inflammatory factors such as TNF-α, or IL1β. Thus, chronic inflammation is present despite viral and immune system restauration.

The high and stable ATP concentrations circulating in HIV-infected individuals indicate that Panx-1 channels are open, but also that purinergic and adenosine signaling is compromised due to the alterations in ATP metabolism. These data show that ATP and its purinergic receptors are essential for HIV entry, and later stages in viral replication may have potential therapeutic implications. The nearly complete inhibition of viral replication by multiple purinergic receptor antagonists suggests that these receptors may be good targets for therapy. In fact, P2X receptors participate in neuropathic pain, inflammatory disease, and potentially depression, and a number of purinergic receptor antagonists are already in testing for human therapy [109-115]. Studies using oATP, which the inventors found to be a potent inhibitor of HIV replication and viral entry, in an in vivo mouse model demonstrated that it is a good systemic blocker of purinergic receptors, which can prevent the onset of diabetes and inflammatory bowel disease [116]. These findings show that preventing ATP accumulation or blocking it signaling reduces the chronic inflammation observed in all HIV-infected individuals and should be considered as potential therapies for HIV.

FIG. 5 is a graph that shows mRNA expression profile of human primary astrocytes, a key component of the human Blood Brain barrier. qRT-PCR determined that P2X4, P2X7, and P2Y1 are the main expressed purinergic receptor in astrocytes. The negative control, PECAM-1 and the positive control Connexin 43, Cx43, are the internal controls. All other receptors were negatives. N=4 different individuals.

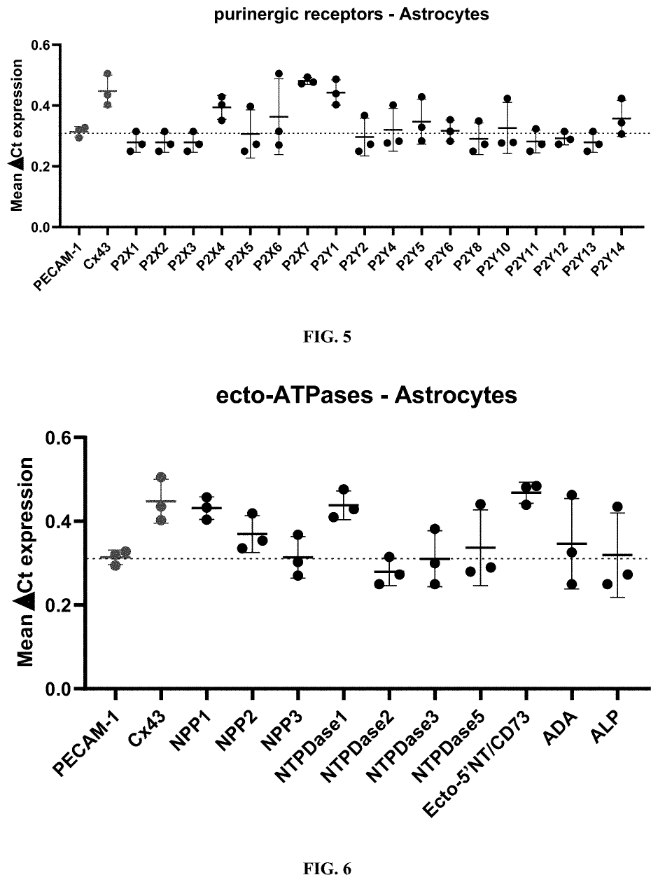

FIG. 6 is a graph that shows mRNA expression of ecto-ATPases on human primary astrocytes, a key component of the blood brain barrier. qRT-PCR determined that NNP1, NNP2, NTPDase 1 and CD73 are the main ecto ATPases expressed in astrocytes. The negative control, PECAM-1 and the positive control Connexin 43, Cx43, are the internal controls. All other receptors were negatives. N=4 different individuals.

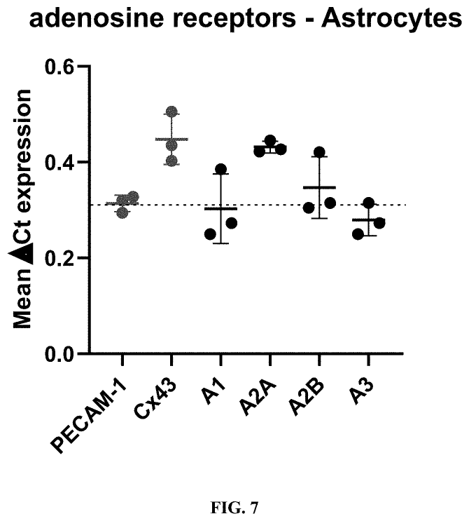

FIG. 7 is a graph that shows mRNA expression of adenosine receptors on human primary astrocytes, a key component of the blood brain barrier. qRT-PCR determined that A2A was the only adenosine receptor expressed in astrocytes. The negative control, PECAM-1 and the positive control Connexin 43, Cx43, are the internal controls. All other receptors were negatives. N=4 different individuals.

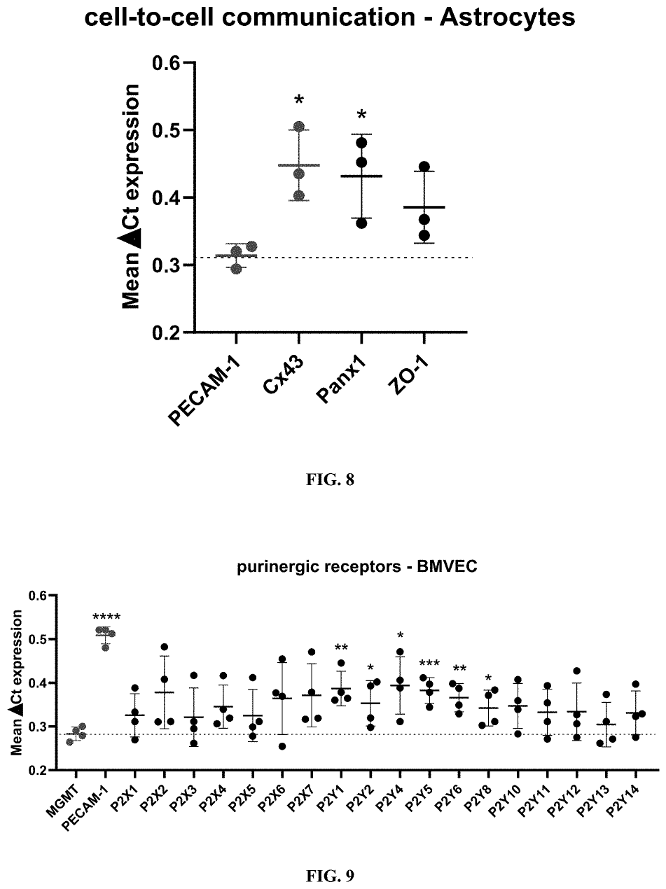

FIG. 8 is a graph that shows mRNA expression of cell to cell communication mRNA required for efficient astrocyte-astrocyte communication. qRT-PCR determined that Cx43, pannexin-1 and ZO-1 are expressed in astrocytes. The negative control, PECAM-1 and the positive control Connexin 43, Cx43, are the internal controls. All other receptors were negatives. N=4 different individuals.

FIG. 9 is a graph that shows mRNA expression profile of human primary brain endothelial cells, a key component of the human Blood Brain barrier. qRT-PCR determined that P2Y1, Y2, Y4, Y5, Y6, and Y8 are the main expressed purinergic receptor in astrocytes. The positive control, PECAM-1 and the positive control MGMT are the internal controls. All other receptors were negatives. N=4 different individuals.

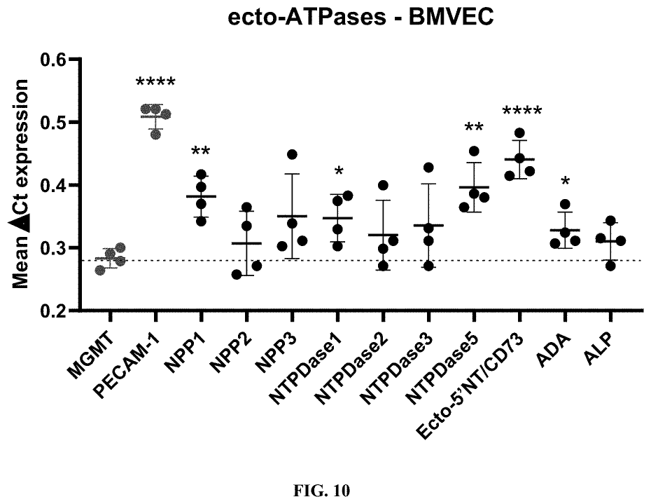

FIG. 10 is a graph that shows mRNA expression of ecto-ATPases on human primary brain endothelial cells, a key component of the blood brain barrier. qRT-PCR determined that NNP1, NTPDase 1, NTPDase 5 and CD73 as well ADA are the main ecto ATPases expressed in human brain endothelial cells. The positive control, PECAM-1 and the positive control MGMT are the internal controls. All other receptors were negatives. N=4 different individuals.

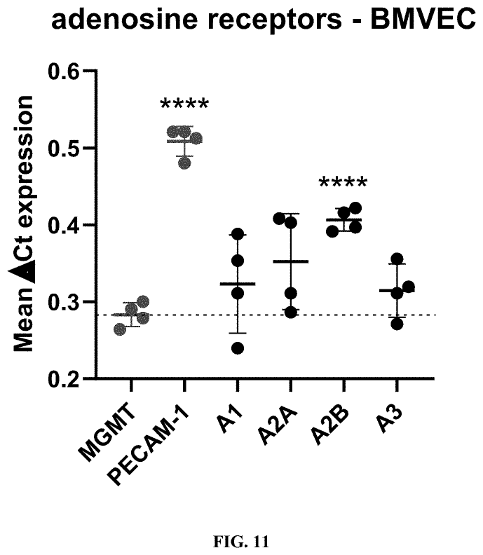

FIG. 11 is a graph that shows mRNA expression of adenosine receptors on human primary brain endothelial cells, a key component of the blood brain barrier. qRT-PCR determined that A2B was the only adenosine receptor expressed in human primary brain endothelial cells. The positive control, PECAM-1 and the positive control MGMT are the internal controls. All other receptors were negatives. N=4 different individuals.

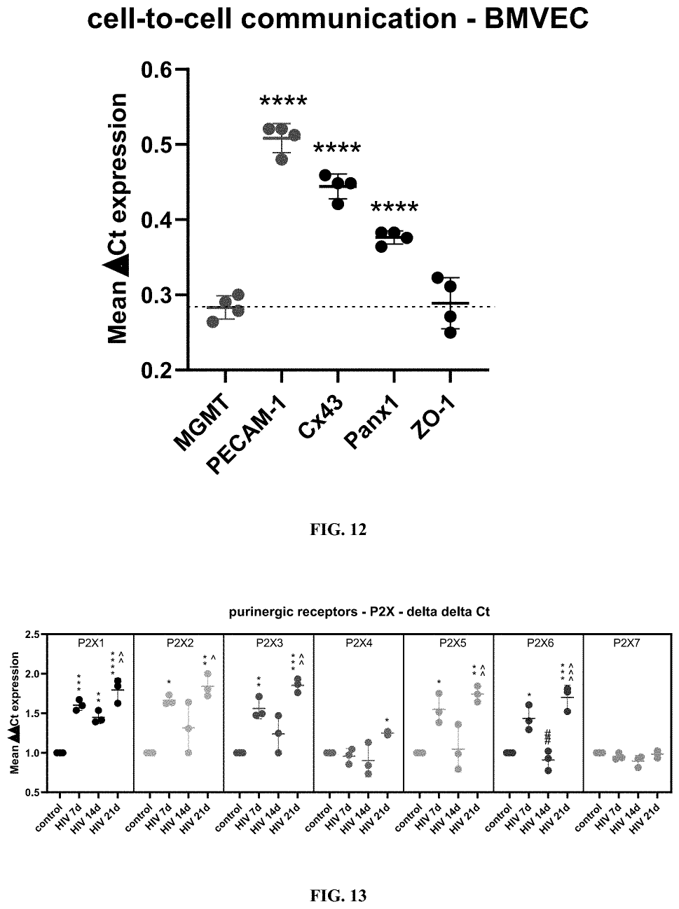

FIG. 12 is a graph that shows mRNA expression of cell to cell communication mRNA required for efficient endothelial cell communication. qRT-PCR determined that Cx43, pannexin-1 and ZO-1 are expressed in on human primary brain endothelial cells. The negative control, PECAM-1 and the positive control Connexin 43, Cx43, are the internal controls. All other receptors were negatives. N=4 different individuals.

FIG. 13 is a graph that shows mRNA expression profile of human primary astrocytes, a key component of the human Blood Brain barrier, after HIV infection. qRT-PCR determined that P2X1, P2X2, P2X3, P2X4, P2X5, P2X6 and P2X7 are affected by HIV infection. The negative control, PECAM-1 and the positive control Connexin 43, Cx43, are the internal controls. All other receptors were negatives. N=4 different individuals.

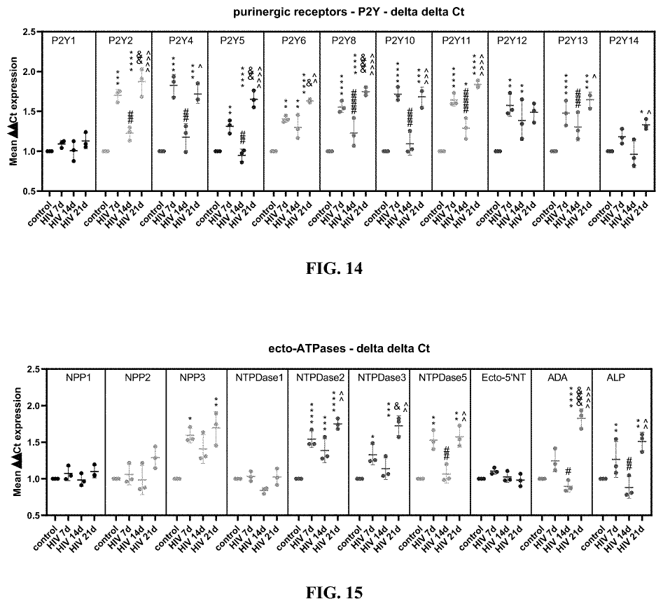

FIG. 14 is a graph that shows mRNA expression profile of human primary astrocytes, a key component of the human Blood Brain barrier, after HIV infection. qRT-PCR determined that P2Y1, Y2, Y4, Y5, Y6, Y8, YI0. Y11, Y12, Y13, and Y14 are altered by HIV infection. The negative control, PECAM-1 and the positive control Connexin 43, Cx43, are the internal controls. All other receptors were negatives. N=4 different individuals.

FIG. 15 is a graph that shows mRNA expression of ecto-ATPases on human primary brain endothelial cells, a key component of the blood brain barrier. qRT-PCR determined that several ecto-ATPases are altered by HIV infection. The positive control, PECAM-1 and the positive control MGMT are the internal controls. All other receptors were negatives. N=4 different individuals.

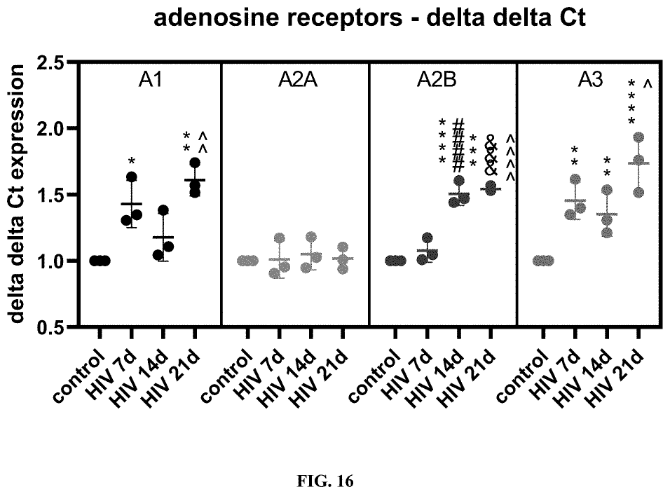

FIG. 16 is a graph that shows mRNA expression of adenosine receptors on human primary brain endothelial cells, a key component of the blood brain barrier. qRT-PCR determined that several adenosine receptors are altered by HIV infection. The positive control, PECAM-1 and the positive control MGMT are the internal controls. All other receptors were negatives. N=4 different individuals.

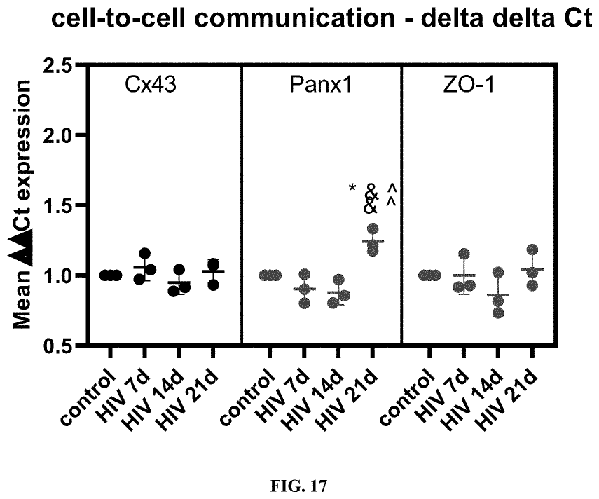

FIG. 17 is a graph that shows mRNA expression of communication systems onhuman primary brain endothelial cells, a key component of the blood brain barrier, are altered upon infection. qRT-PCR determined that several communication systems are altered by HIV infection. The positive control, PECAM-1 and the positive control MGMT are the internal controls. All other receptors were negatives. N=4 different individuals.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±0.1, 0.5, 1, 2, 3, 4, 5, 6, 7, 10, 12 or 15%, or as understood to be within a normal tolerance in the art, for example, within 2 standard deviations of the mean. Unless otherwise clear from the context, all numerical values provided herein are modified by the term about.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f) or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Churchill M J, Deeks S G, Margolis D M, Siliciano R F, Swanstrom R. HIV reservoirs: what, where and how to target them. Nat Rev Microbiol. 2016; 14(1): 55-60.
2. Walker B D, Yu X G. Unravelling the mechanisms of durable control of HIV-1. Nat Rev Immunol. 2013; 13(7): 487-98.
3. Zhou H, Xu M, Huang Q, Gates A T, Zhang X D, Castle J C, et al. Genome-scale RNAi screen for host factors required for HIV replication. Cell Host Microbe. 2008; 4(5): 495-504.
4. Valdebenito S, Barreto A, Eugenin E A. The role of connexin and pannexin containing channels in the innate and acquired immune response. Biochim Biophys Acta. 2018; 1860(1): 154-65.
5. Malik S, Eugenin E A. Role of Connexin and Pannexin containing channels in HIV infection and NeuroAlDS. Neurosci Lett. 2017.
6. Velasquez S, Malik S, Lutz S E, Scemes E, Eugenin E A. Pannexin1 Channels Are Required for Chemokine-Mediated Migration of CD4+T Lymphocytes: Role in Inflammation and Experimental Autoimmune Encephalomyelitis. J Immunol. 2016; 196(10): 4338-47.
7. Malik S, Eugenin E A. Mechanisms of HIV Neuropathogenesis: Role of Cellular Communication Systems. Curr HIV Res. 2016; 14(5): 400-11.
8. Velasquez S, Eugenin E A. Role of Pannexin-1 hemichannels and purinergic receptors in the pathogenesis of human diseases. Front Physiol. 2014; 5: 96.
9. Seror C, Melki M T, Subra F, Raza S Q. Bras M, Saidi H, et al. Extracellular ATP acts on P2Y2 purinergic receptors to facilitate HIV-1 infection. J Exp Med. 2011; 208(9): 1823-34.
10. Velasquez S. M S, Lutz S., Scemes E., and Eugenin E. A. Pannexin1 Channels Are Required for Chemokine-Mediated Migration of CD4+T Lymphocytes: Role in Inflammation and Experimental Autoimmune Encephalomyelitis. The Journal of Immunology. 2016; In press.
11. Subbian S, Eugenin E, Kaplan G. Detection of Mycobacterium tuberculosis in latently infected lungs by immunohistochemistry and confocal microscopy. J Med Microbiol. 2014; 63(Pt 11): 1432-5.
12. Orellana J A, Velasquez S, Williams D W, Saez J C, Berman J W, Eugenin E A. Pannexin1 hemichannels are critical for HIV infection of human primary CD4+T lymphocytes. Journal of leukocyte biology. 2013; 94(3): 399-407.
13. Hazleton J E, Berman J W, Eugenin E A. Purinergic receptors are required for HIV-1 infection of primary human macrophages. J Immunol. 2012; 188(9): 4488-95.
14. Jernigan T L, Archibald S L, Fennema-Notestine C, Taylor M J, Theilmann R J, Julaton M D, et al. Clinical factors related to brain structure in HIV: the CHARTER study. Journal of neurovirology. 2011; 17(3): 248-57.
15. Heaton R K, Clifford D B, Franklin D R, Jr., Woods S P, Ake C, Vaida F, et al. HIV-associated neurocognitive disorders persist in the era of potent antiretroviral therapy: CHARTER Study. Neurology. 2010; 75(23): 2087-96.
16. Woods S P, Rippeth J D, Frol A B, Levy J K, Ryan E, Soukup V M, et al. Interrater reliability of clinical ratings and neurocognitive diagnoses in HIV. J Clin Exp Neuropsychol. 2004; 26(6): 759-78.
17. Eugenin E A, Berman J W. Chemokine-dependent mechanisms of leukocyte trafficking across a model of the blood-brain barrier. Methods. 2003; 29(4). 351-61.
18. Hurwitz A A, Berman J W, Lyman W D. The role of the blood-brain barrier in HIV infection of the central nervous system. Adv Neuroimmunol. 1994; 4(3): 249-56.
19. Hurwitz A A, Berman J W, Rashbaum W K, Lyman W D. Human fetal astrocytes induce the expression of blood-brain barrier specific proteins by autologous endothelial cells. Brain Res. 1993; 625(2): 238-43.
20. Weiss J M, Downie S A, Lyman W D, Berman J W. Astrocyte-derived monocyte-chemoattractant protein-1 directs the transmigration of leukocytes across a model of the human blood-brain barrier. J Immunol. 1998; 161(12): 6896-903.
21. Eugenin E A, Berman J W. Gap junctions mediate human immunodeficiency virus-bystander killing in astrocytes. J Neurosci. 2007; 27(47): 12844-50.
22. Eugenin E A, Osiecki K, Lopez L, Goldstein H, Calderon™, Berman J W. CCL2/monocyte chemoattractant protein-1 mediates enhanced transmigration of human immunodeficiency virus (HIV)-infected leukocytes across the blood-brain barrier: a potential mechanism of HIV-CNS invasion and NeuroAIDS. J Neurosci. 2006; 26(4): 1098-106.
23. Seror C, Melki M T, Subra F, Raza S Q, Bras M, Saidi H, et al. Extracellular ATP acts on P2Y2 purinergic receptors to facilitate HIV-1 infection. J Exp Med. 2011; 208(9): 1823-34.
24. Orellana J A, Froger N, Ezan P, Jiang J X, Bennett M V, Naus C C, et al. ATP and glutamate released via astroglial connexin 43 hemichannels mediate neuronal death through activation of pannexin 1 hemichannels. Journal of neurochemistry. 2011.
25. Sanchez H A, Orellana J A, Verselis V K, Saez J C. Metabolic inhibition increases activity of connexin-32 hemichannels permeable to Ca2+ in transfected HeLa cells. American journal of physiology Cell physiology. 2009; 297(3): C665-78.
26. Pelegrin P, Surprenant A. Pannexin-1 mediates large pore formation and interleukin-1beta release by the ATP-gated P2X7 receptor. Embo J. 2006; 25(21): 5071-82.
27. Silverman W, Locovei S, Dahl G. Probenecid, a gout remedy, inhibits pannexin 1 channels. Am J Physiol Cell Physiol. 2008; 295(3). C761-7.
28. Evans W H, De Vuyst E, Leybaert L. The gap junction cellular internet: connexin hemichannels enter the signalling limelight. Biochem J. 2006; 397(1): 1-14.
29. Siller-Jackson A J, Burra S, Gu S, Xia X, Bonewald L F, Sprague E, et al. Adaptation of connexin 43-hemichannel prostaglandin release to mechanical loading. The Journal of biological chemistry. 2008; 283(39): 26374-82.
30. Burnstock G. Do some nerve cells release more than one transmitter?Neuroscience. 1976; 1(4): 239-48.
31. Elliott M R, Chekeni F B, Trampont P C, Lazarowski E R, Kadi A, Walk S F, et al. Nucleotides released by apoptotic cells act as a find-me signal to promote phagocytic clearance. Nature. 2009; 461(7261): 282-6.
32. Ghiringhelli F, Apetoh L, Tesniere A, Aymeric L, Ma Y, Ortiz C, et al. Activation of the NLRP3 inflammasome in dendritic cells induces IL-1beta-dependent adaptive immunity against tumors. Nature medicine. 2009; 15(10): 1170-8.
33. Locovei S, Bao L, Dahl G. Pannexin 1 in erythrocytes: function without a gap. Proceedings of the National Academy of Sciences of the United States of America. 2006; 103(20): 7655-9.
34. Locovei S, Scemes E, Qiu F, Spray D C, Dahl G. Pannexin1 is part of the pore forming unit of the P2X(7) receptor death complex. FEBS letters. 2007; 581(3): 483-8.
35. Di Virgilio F. Dr. Jekyll/Mr. Hyde: the dual role of extracellular ATP. Journal of the autonomic nervous system. 2000; 81(1-3): 59-63.
36. Gorman M W, Feigl E O, Buffington C W. Human plasma ATP concentration. Clin Chem. 2007; 53(2): 318-25.
37. Calderon™, Williams D W, Lopez L, Eugenin E A, Cheney L, Gaskill P J, et al. Dopamine Increases CD14(+)CD16(+) Monocyte Transmigration across the Blood Brain Barrier: Implications for Substance Abuse and HIV Neuropathogenesis. J Neuroimmune Pharmacol. 2017; 12(2): 353-70.
38. Williams D W, Calderon™, Lopez L, Carvallo-Torres L, Gaskill P J, Eugenin E A, et al. Mechanisms of HIV entry into the CNS: increased sensitivity of HIV infected CD14+CD16+ monocytes to CCL2 and key roles of CCR2, JAM-A, and ALCAM in diapedesis. PLoS One. 2013; 8(7): e69270.
39. Roberts T K, Eugenin E A, Lopez L, Romero I A, Weksler B B, Couraud P O, et al. CCL2 disrupts the adherens junction: implications for neuroinflammation. Lab Invest. 2012; 92(8): 1213-33.
40. Eugenin E A, Clements J E, Zink M C, Berman J W. Human immunodeficiency virus infection of human astrocytes disrupts blood-brain barrier integrity by a gap junction-dependent mechanism. J Neurosci. 2011; 31(26): 9456-65.
41. Williams D W, Veenstra M, Gaskill P J, Morgello S, Calderon™, Berman J W. Monocytes mediate HIV neuropathogenesis: mechanisms that contribute to HIV associated neurocognitive disorders. Curr HIV Res. 2014; 12(2): 85-96.
42. Mankowski J L, Queen S E, Kirstein L M, Spelman J P, Laterra J, Simpson I A, et al. Alterations in blood-brain barrier glucose transport in SIV-infected macaques. J Neurovirol. 1999; 5(6): 695-702.
43. Roberts T K, Eugenin E A, Lopez L, Romero I A, Weksler B B, Couraud P O, et al. CCL2 disrupts the adherens junction: implications for neuroinflammation. Lab Invest. 2012.
44. McLaurin K A, Li H, Booze R M, Mactutus C F. Disruption of Timing: NeuroHIV Progression in the Post-cART Era. Sci Rep. 2019; 9(1): 827.
45. Heithoff A J, Totusek S A, Le D, Barwick L, Gensler G, Franklin D R, et al. The integrated National NeuroAIDS Tissue Consortium database: a rich platform for neuroHIV research. Database (Oxford). 2019; 2019.
46. Barat C, Proust A, Deshiere A, Leboeuf M, Drouin J, Tremblay M J. Astrocytes sustain long-term productive HIV-1 infection without establishment of reactivable viral latency. Glia. 2018; 66(7): 1363-81.
47. Surdo M, Cortese M F, Perno C F, Aquaro S. NeuroAiDS: virological aspects of HIV infection. J Biol Regul Homeost Agents. 2013; 27(2 Suppl): 115-28.
48. Kolson D L, Lavi E, Gonzalez-Scarano F. The effects of human immunodeficiency virus in the central nervous system. Adv Virus Res. 1998; 50: 1-47.
49. Shakirzyanova M, Kong X P, Cheng-Mayer C. Determinants of HIV-1 CD4-Independent Brain Adaptation. J Acquir Immune Defic Syndr. 2017; 76(2): 209-18.
50. Marban C, Forouzanfar F, Ait-Ammar A, Fahmi F, El Mekdad H, Daouad F, et al. Targeting the Brain Reservoirs: Toward an HIV Cure. Front Immunol. 2016; 7: 397.
51. Cary D C, Fujinaga K, Peterlin B M. Molecular mechanisms of HIV latency. J Clin Invest. 2016; 126(2): 448-54.
52. Brew B J, Robertson K, Wright E J, Churchill M, Crowe S M, Cysique L A, et al. HIV eradication symposium: will the brain be left behind? J Neurovirol. 2015; 21(3): 322-34.
53. Hermansson L, Yilmaz A, Axelsson M, Blennow K, Fuchs D, Hagberg L, et al. Cerebrospinal fluid levels of glial marker YKL-40 strongly associated with axonal injury in HIV infection. J Neuroinflammation. 2019; 16(1): 16.
54. McLaurin K A, Booze R M, Mactutus C F. Diagnostic and prognostic biomarkers for HAND. J Neurovirol. 2019.
55. Jessen Krut J, Mellberg T, Price R W, Hagberg L, Fuchs D, Rosengren L, et al. Biomarker evidence of axonal injury in neuroasymptomatic HIV-1 patients. PLoS One. 2014; 9(2): e88591.
56. Cysique L A, Juge L, Lennon M J, Gates™, Jones S P, Lovelace M D, et al. HIV brain latency as measured by CSF BcL11b relates to disrupted brain cellular energy in virally suppressed HIV infection. AIDS. 2019; 33(3): 433-41.
57. Eden A, Marcotte T D, Heaton R K, Nilsson S, Zetterberg H, Fuchs D, et al. Increased Intrathecal Immune Activation in Virally Suppressed HIV-1 Infected Patients with Neurocognitive Impairment. PLoS One. 2016; 11(6): e0157160.
58. Gisslen M, Heslegrave A, Veleva E, Yilmaz A, Andersson L M, Hagberg L, et al. CSF concentrations of soluble TREM2 as a marker of microglial activation in HIV-1 infection. Neurol Neuroimmunol Neuroinflamm. 2019; 6(1): e512.
59. Guha D, Mukerji S S, Chettimada S, Misra V, Lorenz D R, Morgello S, et al.
Cerebrospinal fluid extracellular vesicles and neurofilament light protein as biomarkers of central nervous system injury in HIV-infected patients on antiretroviral therapy. AIDS. 2019; 33(4): 615-25.
60. Van Zoest R A, Underwood J, De Francesco D, Sabin C A, Cole J H, Wit F W, et al. Structural brain abnormalities in successfully treated HIV infection: associations with disease and cerebrospinal fluid biomarkers. J Infect Dis. 2017.
61. Andreoni M, Mussi C, Bellagamba R, Di Campli F, Montinaro V, Babiloni C. Biomarkers of monitoring and functional reserve of physiological systems over time in HIV: expert opinions for effective secondary prevention. New Microbiol. 2017; 40(4).
62. Mehta S R, Perez-Santiago J, Hulgan T, Day T R, Barnholtz-Sloan J, Gittleman H, et al. Cerebrospinal fluid cell-free mitochondrial DNA is associated with HIV replication, iron transport, and mild HIV-associated neurocognitive impairment. J Neuroinflammation. 2017; 14(1): 72.
63. Hellmuth J, Fletcher J L, Valcour V, Kroon E, Ananworanich J, Intasan J, et al. Neurologic signs and symptoms frequently manifest in acute HIV infection. Neurology. 2016; 87(2): 148-54.
64. Anderson A M, Harezlak J, Bharti A, Mi D, Taylor M J, Daar E S, et al. Plasma and Cerebrospinal Fluid Biomarkers Predict Cerebral Injury in HIV-Infected Individuals on Stable Combination Antiretroviral Therapy. J Acquir Immune Defic Syndr. 2015; 69(1): 29-35.
65. Harezlak J, Cohen R, Gongvatana A, Taylor M, Buchthal S, Schifitto G, et al. Predictors of CNS injury as measured by proton magnetic resonance spectroscopy in the setting of chronic HIV infection and CART. J Neurovirol. 2014; 20(3): 294-303.
66. Bairwa D, Kumar V, Vyas S, Das B K, Srivastava A K, Pandey R M, et al. Case control study: magnetic resonance spectroscopy of brain in HIV infected patients. BMC Neurol. 2016; 16: 99.
67. Chong W K, Sweeney B, Wilkinson I D, Paley M, Hall-Craggs M A, Kendall B E, et al. Proton spectroscopy of the brain in HIV infection: correlation with clinical, immunologic, and M R imaging findings. Radiology. 1993; 188(1): 119-24.
68. Laubenberger J, Haussinger D, Bayer S, Thielemann S, Schneider B, Mundinger A, et al. HIV-related metabolic abnormalities in the brain: depiction with proton M R spectroscopy with short echo times. Radiology. 1996; 199(3): 805-10.
69. Suwanwelaa N, Phanuphak P, Phanthumchinda K, Suwanwela N C, Tantivatana J, Ruxrungtham K, et al. Magnetic resonance spectroscopy of the brain in neurologically asymptomatic HIV-infected patients. Magn Reson Imaging. 2000; 18(7): 859-65.
70. Castellano P, Prevedel L, Valdebenito S, Eugenin E A. HIV infection and latency induce a unique metabolic signature in human macrophages. Sci Rep. 2019; 9(1): 3941.
71. Sailasuta N, Ananworanich J, Lerdlum S, Sithinamsuwan P, Fletcher J L, Tipsuk S, et al. Neuronal-Glia Markers by Magnetic Resonance Spectroscopy in HIV Before and After Combination Antiretroviral Therapy. J Acquir Immune Defic Syndr. 2016; 71(1): 24-30.
72. Wright P W, Vaida F F, Fernandez R J, Rutlin J, Price R W, Lee E, et al. Cerebral white matter integrity during primary HIV infection. AIDS. 2015; 29(4): 433-42.
73. Drewes J L, Meulendyke K A, Liao Z, Witwer K W, Gama L, Ubaida-Mohien C, et al. Quinolinic acid/tryptophan ratios predict neurological disease in SIV-infected macaques and remain elevated in the brain under cART. J Neurovirol. 2015; 21(4): 449-63.
74. Dickens A M, Anthony D C, Deutsch R, Mielke M M, Claridge T D, Grant I, et al.
Cerebrospinal fluid metabolomics implicate bioenergetic adaptation as a neural mechanism regulating shifts in cognitive states of HIV-infected patients. AIDS. 2015; 29(5): 559-69.
75. Scemes E, Spray D C, Meda P. Connexins, pannexins, innexins: novel roles of “hemi-channels”. Pflugers Archiv: European journal of physiology. 2009; 457(6): 1207-26.
76. Boassa D, Ambrosi C, Qiu F, Dahl G, Gaietta G, Sosinsky G. Pannexin1 channels contain a glycosylation site that targets the hexamer to the plasma membrane. The Journal of biological chemistry. 2007; 282(43): 31733-43.
77. Penuela S, Bhalla R, Gong X Q, Cowan K N, Celetti S J, Cowan B J, et al. Pannexin 1 and pannexin 3 are glycoproteins that exhibit many distinct characteristics from the connexin family of gap junction proteins. Journal of cell science. 2007; 120(Pt 21): 3772-83.
78. Celetti S J, Cowan K N, Penuela S, Shao Q, Churko J, Laird D W. Implications of pannexin 1 and pannexin 3 for keratinocyte differentiation. Journal of cell science. 2010; 123(Pt 8): 1363-72.
79. Kurtenbach S, Prochnow N, Kurtenbach S, Klooster J, Zoidl C, Dermietzel R, et al. Pannexin1 channel proteins in the zebrafish retina have shared and unique properties. PloS one. 2013; 8(10): e77722.
80. Penuela S, Bhalla R, Nag K, Laird D W. Glycosylation regulates pannexin intermixing and cellular localization. Molecular biology of the cell. 2009, 20(20): 4313-23.
81. Qu Y, Misaghi S, Newton K, Gilmour L L, Louie S, Cupp J E, et al. Pannexin-1 is required for ATP release during apoptosis but not for inflammasome activation. J Immunol. 2011; 186(11): 6553-61.
82. Chekeni F B, Elliott M R, Sandilos J K, Walk S F, Kinchen J M, Lazarowski E R, et al. Pannexin 1 channels mediate ‘find-me’ signal release and membrane permeability during apoptosis. Nature. 2010; 467(7317): 863-7.
83. Forsyth A M, Wan J, Owrutsky P D, Abkarian M, Stone H A. Multiscale approach to link red blood cell dynamics, shear viscosity, and ATP release. Proceedings of the National Academy of Sciences of the United States of America. 2011; 108(27): 10986-91.
84. Antonioli L, Colucci R, Pellegrini C, Giustarini G, Tuccori M, Blandizzi C, et al. The role of purinergic pathways in the pathophysiology of gut diseases: pharmacological modulation and potential therapeutic applications. Pharmacol Ther. 2013; 139(2): 157-88.
85. Antonioli L, Pacher P, Vizi E S, Hasko G. CD39 and CD73 in immunity and inflammation. Trends Mol Med. 2013; 19(6): 355-67.
86. Shapiro M J, Jellinek M, Pyrros D, Sundine M, Baue A E. Clearance and maintenance of blood nucleotide levels with adenosine triphosphate-magnesium chloride injection. Circ Shock. 1992; 36(1): 62-7.
87. Ehrentraut H, Westrich J A, Eltzschig H K, Clambey E T. Adora2b adenosine receptor engagement enhances regulatory T cell abundance during endotoxin-induced pulmonary inflammation. PLoS One. 2012; 7(2): e32416.
88. Colgan S P, Eltzschig H K. Adenosine and hypoxia-inducible factor signaling in intestinal injury and recovery. Annu Rev Physiol. 2012; 74: 153-75.
89. Zhou F, Liu X, Gao L, Zhou X, Cao Q, Niu L, et al. HIV-1 Tat enhances purinergic P2Y4 receptor signaling to mediate inflammatory cytokine production and neuronal damage via PI3K/Akt and ERK MAPK pathways. J Neuroinflammation. 2019; 16(1): 71.
90. Soare A Y, Durham N D, Gopal R, Tweel B, Hoffman K W, Brown J A, et al. P2X Antagonists Inhibit HIV-1 Productive Infection and Inflammatory Cytokines Interleukin-10 (IL-10) and IL-1beta in a Human Tonsil Explant Model. J Virol. 2019; 93(1).
91. Rezer J F P, Adefegha S A, Ecker A, Passos D F, Saccol R S P, Bertoldo T M D, et al. Changes in inflammatory/cardiac markers of HIV positive patients. Microb Pathog. 2018; 114: 264-8.
92. Yi Z, Xie L, Zhou C, Yuan H, Ouyang S, Fang Z, et al. P2Y12 receptor upregulation in satellite glial cells is involved in neuropathic pain induced by HIV glycoprotein 120 and 2′,3′-dideoxycytidine. Purinergic Signal. 2018; 14(1): 47-58.
93. Jones K R, Choi U, Gao J L, Thompson R D, Rodman L E, Malech H L, et al. A Novel Method for Screening Adenosine Receptor Specific Agonists for Use in Adenosine Drug Development. Sci Rep. 2017; 7: 44816.
94. Menkova-Garnier I, Hocini H, Foucat E, Tisserand P, Bourdery L, Delaugerre C, et al. P2X7 Receptor Inhibition Improves CD34 T-Cell Differentiation in HIV-Infected Immunological Nonresponders on c-ART. PLoS Pathog. 2016; 12(4): e1005571.
95. Swartz T H, Esposito A M, Durham N D, Hartmann B M, Chen B K. P2X-selective purinergic antagonists are strong inhibitors of HIV-1 fusion during both cell-to-cell and cell-free infection. J Virol. 2014; 88(19): 11504-15.
96. Schenk U, Westendorf A M, Radaelli E, Casati A, Ferro M, Fumagalli M, et al. Purinergic control of T cell activation by ATP released through pannexin-1 hemichannels. Sci Signal. 2008; 1(39): ra6.

### ADDITIONAL REFERENCES

1. Velasquez, S., et al., Circulating levels of ATP is a biomarker of HIV cognitive impairment. EBioMedicine, 2019.
2. Malik, S. and E. A. Eugenin, Role of Connexin and Pannexin containing channels in HIV infection and NeuroAIDS. Neurosci Lett, 2019. 695: p. 86-90.
3. Valdebenito, S., et al., The Novel Roles of Connexin Channels and Tunneling Nanotubes in Cancer Pathogenesis. Int J Mol Sci, 2018. 19(5).
4. Hazleton, J. E., J. W. Berman, and E. A. Eugenin, Purinergic receptors are required for HIV-1 infection of primary human macrophages. J Immunol, 2012. 188(9): p. 4488-95.
5. Miyoshi, H., et al., Stable and efficient gene transfer into the retina using an HIV-based lentiviral vector. Proc Natd Acad Sci USA, 1997. 94(19): p. 10319-23.
6. Takahashi, R. H., et al., Analysis of human lymphotropic T-cell virus type I I-like particle production by recombinant baculovirus-infected insect cells. Virology, 1999. 256(2): p. 371-80.
7. Zyma, M. and R. Pawliczak, Characteristics and the role of purinergic receptors in pathophysiology with focus on immune response. Int Rev Immunol, 2020: p. 1-21.
8. Giuliani, A. L., A. C. Sarti, and F. Di Virgilio, Extracellular nucleotides and nucleosides as signalling molecules. Immunol Lett, 2019. 205: p. 16-24.
9. Schmid, R. and R. J. Evans, ATP-Gated P2X Receptor Channels: Molecular Insights into Functional Roles. Annu Rev Physiol, 2019. 81: p. 43-62.
10. Young, C. N. J. and D. C. Gorecki, P2RX7 Purinoceptor as a Therapeutic Target—The Second Corning? Front Chem, 2018. 6: p. 248.
11. Makarenkova, H. P., S. B. Shah, and V. I. Shestopalov, The two faces of pannexins: new roles in inflammation and repair. J Inflamm Res, 2018. 11: p. 273-288.
12. Dosch, M., et al., Mechanisms of ATP Release by Inflammatory Cells. Int J Mol Sci, 2018. 19(4).
13. Dal Ben, D., et al., Purinergic Ligands as Potential Therapeutic Tools for the Treatment of Inflammation-Related Intestinal Diseases. Front Pharmacol, 2018. 9: p. 212.
14. Savio, L. E. B., et al., The P2X7 Receptor in Inflammatory Diseases: Angel or Demon? Front Pharmacol, 2018. 9: p. 52.
15. Wei, L., et al., ATP-activated P2X7 receptor in the pathophysiology of mood disorders and as an emerging target for the development of novel antidepressant therapeutics. Neurosci Biobehav Rev, 2018. 87: p. 192-205.
16. Dou, L., et al., Extracellular ATP signaling and clinical relevance. Clin Immunol, 2018. 188: p. 67-73.
17. Tozzi, M. and I. Novak, Purinergic Receptors in Adipose Tissue As Potential Targets in Metabolic Disorders. Front Pharmacol, 2017. 8: p. 878.
18. Adiele, R. C. and C. A. Adiele, Metabolic defects in multiple sclerosis. Mitochondrion, 2019. 44: p. 7-14.
19. Burnstock, G., K. A. Jacobson, and F. L. Christofi, Purinergic drug targets for gastrointestinal disorders. Curr Opin Pharmacol, 2017. 37: p. 131-141.
20. Burnstock, G., Purinergic Signalling: Therapeutic Developments. Front Pharmacol, 2017. 8: p. 661.
21. Perl, A., Review: Metabolic Control of Immune System Activation in Rheumatic Diseases. Arthritis Rheumatol, 2017. 69(12): p. 2259-2270.
22. Churchill, M. J., et al., HIV reservoirs: what, where and how to target them. Nat Rev Microbiol, 2016. 14(1): p. 55-60.
23. Walker, B. D. and X. G. Yu, Unravelling the mechanisms of durable control of HIV-1. Nat Rev Immunol, 2013. 13(7): p. 487-98.
24. Zhou, H., et al., Genome-scale RNAi screen for host factors required for HIV replication. Cell Host Microbe, 2008. 4(5): p. 495-504.
25. Valdebenito, S., A. Barreto, and E. A. Eugenin, The role of connexin and pannexin containing channels in the innate and acquired immune response. Biochim Biophys Acta, 2018. 1860(1): p. 154-165.
26. Malik, S. and E. A. Eugenin, Role of Connexin and Pannexin containing channels in HIV infection and NeuroAIDS. Neurosci Lett, 2017.
27. Velasquez, S., et al., Pannexin1 Channels Are Required for Chemokine-Mediated Migration of CD4+T Lymphocytes: Role in Inflammation and Experimental Autoimmune Encephalomyelitis. J Immunol, 2016. 196(10): p. 4338-47.
28. Malik, S. and E. A. Eugenin, Mechanisms of HIV Neuropathogenesis: Role of Cellular Communication Systems. Curr HIV Res, 2016. 14(5): p. 400-411.
29. Velasquez, S. and E. A. Eugenin, Role of Pannexin-1 hemichannels and purinergic receptors in the pathogenesis of human diseases. Front Physiol, 2014. 5: p. 96.
30. Seror, C., et al., Extracellular ATP acts on P2Y2 purinergic receptors to facilitate HIV-1 infection. The Journal of experimental medicine, 2011. 208(9): p. 1823-34.
31. Velasquez S., M. S., Lutz S., Scemes E., and Eugenin E. A., Pannexin1 Channels Are Required for Chemokine-Mediated Migration of CD4+T Lymphocytes: Role in Inflammation and Experimental Autoimmune Encephalomyelitis. The Journal of Immunology, 2016. In press.
32. Subbian, S., E. Eugenin, and G. Kaplan, Detection of Mycobacterium tuberculosis in latently infected lungs by immunohistochemistry and confocal microscopy. J Med Microbiol, 2014. 63(Pt 11): p. 1432-5.
33. Orellana, J. A., et al., Pannexin1 hemichannels are critical for HIV infection of human primary CD4+T lymphocytes. J Leukoc Biol, 2013. 94(3): p. 399-407.
34. Jernigan, T. L., et al., Clinical factors related to brain structure in HIV: the CHARTER study. Journal of neurovirology, 2011. 17(3): p. 248-57.
35. Heaton, R. K., et al., HIV-associated neurocognitive disorders persist in the era of potent antiretroviral therapy: CHARTER Study. Neurology, 2010. 75(23): p. 2087-96.
36. Woods, S. P., et al., Interrater reliability of clinical ratings and neurocognitive diagnoses in HIV. J Clin Exp Neuropsychol, 2004. 26(6): p. 759-78.
37. Eugenin, E. A. and J. W. Berman, Chemokine-dependent mechanisms of leukocyte trafficking across a model of the blood-brain barrier. Methods, 2003. 29(4): p. 351-61.
38. Hurwitz, A. A., J. W. Berman, and W. D. Lyman, The role of the blood-brain barrier in HIV infection of the central nervous system. Adv Neuroimmunol, 1994. 4(3): p. 249-56.
39. Hurwitz, A. A., et al., Human fetal astrocytes induce the expression of blood-brain barrier specific proteins by autologous endothelial cells. Brain Res, 1993. 625(2): p. 238-43.
40. Weiss, J. M., et al., Astrocyte-derived monocyte-chemoattractant protein-1 directs the transmigration of leukocytes across a model of the human blood-brain barrier. J Immunol, 1998. 161(12): p. 6896-903.
41. Eugenin, E. A. and J. W. Berman, Gap junctions mediate human immunodeficiency virus-bystander killing in astrocytes. J Neurosci, 2007. 27(47): p. 12844-50.
42. Eugenin, E. A., et al., CCL2/monocyte chemoattractant protein-1 mediates enhanced transmigration of human immunodeficiency virus (HIV)-infected leukocytes across the blood-brain barrier: a potential mechanism of HIV-CNS invasion and NeuroAIDS. J Neurosci, 2006. 26(4): p. 1098-106.
43. Seror, C., et al., Extracellular ATP acts on P2Y2 purinergic receptors to facilitate HIV-1 infection. J Exp Med, 2011. 208(9): p. 1823-34.
44. Orellana, J. A., et al., ATP and glutamate released via astroglial connexin 43 hemichannels mediate neuronal death through activation of pannexin 1 hemichannels. Journal of neurochemistry, 2011.
45. Sanchez, H. A., et al., Metabolic inhibition increases activity of connexin-32 hemichannels permeable to Ca2+ in transfected HeLa cells. American journal of physiology.
Cell physiology, 2009. 297(3): p. C665-78.
46. Pelegrin, P. and A. Surprenant, Pannexin-1 mediates large pore formation and interleukin-1beta release by the ATP-gated P2X7 receptor. The EMBO journal, 2006. 25(21): p. 5071-82.
47. Silverman, W., S. Locovei, and G. Dahl, Probenecid, a gout remedy, inhibits pannexin 1 channels. American journal of physiology. Cell physiology, 2008. 295(3): p. C761-7.
48. Evans, W. H., E. De Vuyst, and L. Leybaert, The gap junction cellular internet. connexin hemichannels enter the signalling limelight. The Biochemical journal, 2006. 397(1): p. 1-14.
49. Siller-Jackson, A. J., et al., Adaptation of connexin 43-hemichannel prostaglandin release to mechanical loading. The Journal of biological chemistry, 2008. 283(39): p. 26374-82.
50. Burnstock, G., Do some nerve cells release more than one transmitter?Neuroscience, 1976. 1(4): p. 239-48.
51. Elliott, M. R., et al., Nucleotides released by apoptotic cells act as a find-me signal to promote phagocytic clearance. Nature, 2009. 461(7261): p. 282-6.
52. Ghiringhelli, F., et al., Activation of the NLRP3 inflammasome in dendritic cells induces IL-1beta-dependent adaptive immunity against tumors. Nat Med, 2009. 15(10): p. 1170-8.
53. Locovei, S., L. Bao, and G. Dahl, Pannexin 1 in erythrocytes: function without a gap. Proc Natl Acad Sci USA, 2006. 103(20): p. 7655-9.
54. Locovei, S., et al., Pannexin1 is part of the pore forming unit of the P2X(7) receptor death complex. FEBS Lett, 2007. 581(3): p. 483-8.
55. Di Virgilio, F., Dr. Jekyll/Mr. Hyde: the dual role of extracellular ATP. J Auton Nerv Syst, 2000. 81(1-3): p. 59-63.
56. Gorman, M. W., E. O. Feigl, and C. W. Buffington, Human plasma ATP concentration. Clin Chem, 2007. 53(2): p. 318-25.
57. Calderon, T. M., et al., Dopamine Increases CD14(+)CD16(+) Monocyte Transmigration across the Blood Brain Barrier: Implications for Substance Abuse and HIV Neuropathogenesis. J Neuroimmune Pharmacol, 2017. 12(2): p. 353-370.
58. Williams, D. W., et al., Mechanisms of HIV entry into the CNS: increased sensitivity of HIV infected CD14+CD16+ monocytes to CCL2 and key roles of CCR2, JAM-A, and ALCAM in diapedesis. PLoS One, 2013. 8(7): p. e69270.
59. Roberts, T. K., et al., CCL2 disrupts the adherens junction: implications for neuroinflammation. Lab Invest, 2012. 92(8): p. 1213-33.
60. Eugenin, E. A., et al., Human immunodeficiency virus infection of human astrocytes disrupts blood-brain barrier integrity by a gap junction-dependent mechanism. J Neurosci, 2011. 31(26): p. 9456-65.
61. Williams, D. W., et al., Monocytes mediate HIV neuropathogenesis: mechanisms that contribute to HIV associated neurocognitive disorders. Curr HIV Res, 2014. 12(2): p. 85-96.
62. Mankowski, J. L., et al., Alterations in blood-brain barrier glucose transport in SIV-infected macaques. J Neurovirol, 1999. 5(6): p. 695-702.
63. Roberts, T. K., et al., CCL2 disrupts the adherens junction: implications for neuroinflammation. Lab Invest, 2012.
64. McLaurin, K. A., et al., Disruption of Timing: NeuroHIV Progression in the Post-cART Era. Sci Rep, 2019. 9(1): p. 827.
65. Heithoff, A. J., et al., The integrated National NeuroAIDS Tissue Consortium database: a rich platform for neuroHIV research. Database (Oxford), 2019. 2019.
66. Barat, C., et al., Astrocytes sustain long-term productive HIV-1 infection without establishment of reactivable viral latency. Glia, 2018. 66(7): p. 1363-1381.
67. Surdo, M., et al., NeuroAIDS: virological aspects of HIV infection. J Biol Regul Homeost Agents, 2013. 27(2 Suppl): p. 115-28.
68. Kolson, D. L., E. Lavi, and F. Gonzalez-Scarano, The effects of human immunodeficiency virus in the central nervous system. Adv Virus Res, 1998. 50: p. 1-47.
69. Shakirzyanova, M., X. P. Kong, and C. Cheng-Mayer, Determinants of HIV-1 CD4-Independent Brain Adaptation. J Acquir Immune Defic Syndr, 2017. 76(2): p. ²⁰⁹-2¹⁸.
70. Marban, C., et al., Targeting the Brain Reservoirs: Toward an HIV Cure. Front Immunol, 2016. 7: p. 397.
71. Cary, D. C., K. Fujinaga, and B. M. Peterlin, Molecular mechanisms of HIV latency. J Clin Invest, 2016. 126(2): p. 448-54.
72. Brew, B. J., et al., HIV eradication symposium: will the brain be left behind? J Neurovirol, 2015. 21(3): p. 322-34.
73. Hermansson, L., et al., Cerebrospinal fluid levels of glial marker YKL-40 strongly associated with axonal injury in HIV infection. J Neuroinflammation, 2019. 16(1): p. 16.
74. McLaurin, K. A., R. M. Booze, and C. F. Mactutus, Diagnostic and prognostic biomarkers for HAND. J Neurovirol, 2019.
75. Jessen Krut, J., et al., Biomarker evidence of axonal injury in neuroasymptomatic HIV-1 patients. PLoS One, 2014. 9(2): p. e88591.
76. Cysique, L. A., et al., HIV brain latency as measured by CSF BcL11b relates to disrupted brain cellular energy in virally suppressed HIV infection. AIDS, 2019. 33(3): p. 433-441.
77. Eden, A., et al., Increased Intrathecal Immune Activation in Virally Suppressed HIV-1 Infected Patients with Neurocognitive Impairment. PLoS One, 2016. 11(6): p. e0157160.
78. Gisslen, M., et al., CSF concentrations of soluble TREM2 as a marker of microglial activation in HIV-1 infection. Neurol Neuroimmunol Neuroinflamm, 2019. 6(1): p. e512.
79. Guha, D., et al., Cerebrospinal fluid extracellular vesicles and neurofilament light protein as biomarkers of central nervous system injury in HIV-infected patients on antiretroviral therapy. AIDS, 2019. 33(4): p. 615-625.
80. Van Zoest, R. A., et al., Structural brain abnormalities in successfully treated HIV infection: associations with disease and cerebrospinal fluid biomarkers. J Infect Dis, 2017.
81. Andreoni, M., et al., Biomarkers of monitoring and functional reserve of physiological systems over time in HIV: expert opinions for effective secondary prevention. New Microbiol, 2017. 40(4).
82. Mehta, S. R., et al., Cerebrospinal fluid cell-free mitochondrial DNA is associated with HIV replication, iron transport, and mild HIV-associated neurocognitive impairment. J Neuroinflammation, 2017. 14(1): p. 72.
83. Hellmuth, J., et al., Neurologic signs and symptoms frequently manifest in acute HIV infection. Neurology, 2016. 87(2): p. 148-54.
84. Anderson, A. M., et al., Plasma and Cerebrospinal Fluid Biomarkers Predict Cerebral Injury in HIV-infected Individuals on Stable Combination Antiretroviral Therapy. J Acquir Immune Defic Syndr, 2015. 69(1): p. 29-35.
85. Harezlak, J., et al., Predictors of CNS injury as measured by proton magnetic resonance spectroscopy in the setting of chronic HIV infection and CART. J Neurovirol, 2014. 20(3): p. 294-303.
86. Bairwa, D., et al., Case control study: magnetic resonance spectroscopy of brain in HIV infected patients. BMC Neurol, 2016. 16: p. 99.
87. Chong, W. K., et al., Proton spectroscopy of the brain in HIV infection: correlation with clinical, immunologic, and M R imaging findings. Radiology, 1993. 188(1): p. 119-24.
88. Laubenberger, J., et al., HIV-related metabolic abnormalities in the brain: depiction with proton M R spectroscopy with short echo times. Radiology, 1996. 199(3): p. 805-10.
89. Suwanwelaa, N., et al., Magnetic resonance spectroscopy of the brain in neurologically asymptomatic HIV-infected patients. Magn Reson Imaging, 2000. 18(7): p. 859-65.
90. Castellano, P., et al., HIV infection and latency induce a unique metabolic signature in human macrophages. Sci Rep, 2019. 9(1): p. 3941.
91. Sailasuta, N., et al., Neuronal-Glia Markers by Magnetic Resonance Spectroscopy in HIV Before and After Combination Antiretroviral Therapy. J Acquir Immune Defic Syndr, 2016. 71(1): p. 24-30.
92. Wright, P. W., et al., Cerebral white matter integrity during primary HIV infection. AIDS, 2015. 29(4): p. 433-42.
93. Drewes, J. L., et al., Quinolinic acid/tryptophan ratios predict neurological disease in SIV-infected macaques and remain elevated in the brain under cART. J Neurovirol, 2015. 21(4): p. 449-63.
94. Dickens, A. M., et al., Cerebrospinal fluid metabolomics implicate bioenergetic adaptation as a neural mechanism regulating shifts in cognitive states of HIV-infected patients. AIDS, 2015. 29(5): p. 559-69.
95. Scemes, E., D. C. Spray, and P. Meda, Connexins, pannexins, innexins: novel roles of “hemi-channels”. Pflugers Arch, 2009. 457(6): p. 1207-26.
96. Boassa, D., et al., Pannexin1 channels contain a glycosylation site that targets the hexamer to the plasma membrane. J Biol Chem, 2007. 282(43): p. 31733-43.
97. Penuela, S., et al., Pannexin 1 and pannexin 3 are glycoproteins that exhibit many distinct characteristics from the connexin family of gap junction proteins. J Cell Sci, 2007. 120(Pt 21): p. ³⁷⁷²-8³.
98. Celetti, S. J., et al., Implications of pannexin 1 and pannexin 3 for keratinocyte differentiation. J Cell Sci, 2010. 123(Pt 8): p. 1363-72.
99. Kurtenbach, S., et al., Pannexin1 channel proteins in the zebrafish retina have shared and unique properties. PLoS One, 2013. 8(10): p. e77722.
100. Penuela, S., et al., Glycosylation regulates pannexin intermixing and cellular localization. Mol Biol Cell, 2009. 20(20): p. 4313-23.
101. Qu, Y., et al., Pannexin-1 is required for ATP release during apoptosis but not for inflammasome activation. J Immunol, 2011. 186(11): p. 6553-61.
102. Chekeni, F. B., et al., Pannexin 1 channels mediate ‘find-me’ signal release and membrane permeability during apoptosis. Nature, 2010. 467(7317): p. 863-7.
103. Forsyth, A. M., et al., Multiscale approach to link red blood cell dynamics, shear viscosity, and ATP release. Proc Natl Acad Sci USA, 2011. 108(27): p. 10986-91.
104. Antonioli, L., et al., The role of purinergic pathways in the pathophysiology of gut diseases. pharmacological modulation and potential therapeutic applications. Pharmacol Ther, 2013. 139(2): p. 157-88.
105. Antonioli, L., et al., CD39 and CD73 in immunity and inflammation. Trends Mol Med, 2013. 19(6): p. 355-67.
106. Shapiro, M. J., et al., Clearance and maintenance of blood nucleotide levels with adenosine triphosphate-magnesium chloride injection. Circ Shock, 1992. 36(1): p. 62-7.
107. Ehrentraut, H., et al., Adora2b adenosine receptor engagement enhances regulatory T cell abundance during endotoxin-induced pulmonary inflammation. PLoS One, 2012. 7(2): p. e32416.
108. Colgan, S. P. and H. K. Eltzschig, Adenosine and hypoxia-inducible factor signaling in intestinal injury and recovery. Annu Rev Physiol, 2012. 74: p. 153-75.
109. Zhou, F., et al., HIV-1 Tat enhances purinergic P2Y4 receptor signaling to mediate inflammatory cytokine production and neuronal damage via PI3K/Akt and ERK MAPK pathways. J Neuroinflammation, 2019. 16(1): p. 71.
110. Soare, A. Y., et al., P2X Antagonists Inhibit HIV-1 Productive Infection and Inflammatory Cytokines Interleukin-10 (IL-10) and IL-1beta in a Human Tonsil Explant Model. J Virol, 2019. 93(1).
111. Rezer, J. F. P., et al., Changes in inflammatory/cardiac markers of HIV positive patients. Microb Pathog, 2018. 114; p. 264-268.
112. Yi, Z., et al., P2Y12 receptor upregulation in satellite glial cells is involved in neuropathic pain induced by HIV glycoprotein 120 and 2′,3′-dideoxycytidine. Purinergic Signal, 2018. 14(1): p. ⁴⁷-5⁸.
113. Jones, K. R., et al., A Novel Method for Screening Adenosine Receptor Specific Agonists for Use in Adenosine Drug Development. Sci Rep, 2017. 7: p. 44816.
114. Menkova-Garnier, I., et al., P2X7 Receptor Inhibition Improves CD34 T-Cell Differentiation in HIV-Infected Immunological Nonresponders on c-ART. PLoS Pathog, 2016. 12(4): p. e1005571.
115. Swartz, T. H., et al., P2X-selective purinergic antagonists are strong inhibitors of HIV-1 fusion during both cell-to-cell and cell-free infection. J Virol, 2014. 88(19): p. 11504-15.
116. Schenk, U., et al., Purinergic control of T cell activation by ATP released through pannexin-1 hemichannels. Sci Signal, 2008. 1(39); p. ra6.
117. Gendelman, Howard E. “Predictive biomarkers for cognitive decline during progressive HIV infection.” EBioMedicine 51 (2020).
118. Gajardo-G6mez, Rosario, et al. “HIV GP120 protein increases the function of connexin 43 hemichannels and pannexin-1 channels in astrocytes: Repercussions on astroglial function.” International journal of molecular sciences 21.7 (2020): 2503.

What is claimed is:

1. A method of treating or preventing a neurodegenerative disease in an subject comprising administering to the subject an effective amount of at least one of: a compound, a peptide, a protein, or a nucleic acid expression vector that expresses a peptide or protein that blocks pannexin-1 opening, secretion or stability of an ATP regulator, or activation of purinergic receptors sufficient to treat or prevent the neurodegenerative disease.

2. The method of claim 1, wherein the nucleic acid expression vector is virus-based nucleic acid expression vector.

3. The method of claim 1, wherein the compound, peptide or protein targets the central nervous system (CNS).

4. The method of claim 1, wherein the nucleic acid expression vector is virus-based nucleic acid expression vector under a specific or unspecific control element that expresses in the CNS.

5. The method of claim 1, wherein the pannexin-1 polypeptide, ATP regulator, or purinergic blockers comprises an amino acid sequence or structure having at least 85% identity to the cited blockers.

6. The method of claim 1, wherein the pannexin-1, ATP enzyme, or purinergic peptides comprises an amino acid sequence having at least about 95% amino acid sequence identity to the original sequence cited above.

7. The method of claim 1, wherein the neurodegenerative disease is selected from at least one of NeuroHIV Disease, Alzheimer's Disease, Parkinson Disease, Amyotrophic lateral sclerosis (ALS), multiple sclerosis (MS), or CNS disease with high circulating levels of ATP.

8. The method of claim 1, wherein the subject is a human.

9. The method of claim 1, wherein the compound, peptide, protein, or nucleic acid expression vector is administered systemically or locally.

10. The method of claim 1, wherein the compound, peptide, protein, or nucleic acid expression vector is targeted to the circulation or cells exposed to the circulation.

11. The method of claim 1, wherein the compound, peptide, protein, or nucleic acid expression vector is targeted to prevent at least one of: blood brain barrier (BBB) overactivation or CNS compromise.

12. A method for identifying a candidate or candidate agents for the treatment or prevention of neurodegenerative diseases based on ATP dysregulation comprising:
determining ATP circulating levels in a subject with cognitive impairment;
determining the effect, if any, of a test agent on at least one of: blood brain barrier (BBB) function, immune activation, inflammation, or CNS compromise; and
providing a single or combination of treatments that reduce circulating levels of ATP and its effects on a human.

13. The method of claim 12, wherein said expression vectors, peptides, or targeted compounds with one or more agents that facilitate the crossing of the BBB.

14. A method for at least one of: screening, preventing release, accumulation, or measuring signaling associated with high levels of circulating ATP in a neurodegenerative diseases comprising obtaining a sample from a subject, detecting levels of ATP as biomarkers of cognitive disease; and preventing or treating the subject with effective amount of at least one of: a compound, a peptide, a protein, or a nucleic acid expression vector that expresses a peptide or protein that blocks pannexin-1 opening, secretion or stability of an ATP regulator, or activation of purinergic receptors sufficient to treat or prevent the neurodegenerative disease.

15. The method of claim 14, wherein the nucleic acid expression vector is virus-based nucleic acid expression vector.

16. The method of claim 14, wherein the compound, peptide or protein targets the central nervous system (CNS).

17. The method of claim 14, wherein the nucleic acid expression vector is virus-based nucleic acid expression vector under a specific or unspecific control element that expresses in the CNS.

18. The method of claim 14, wherein the pannexin-1 polypeptide, ATP regulator, or purinergic blockers comprises an amino acid sequence or structure having at least 85% identity to the cited blockers.

19. The method of claim 14, wherein the neurodegenerative disease is selected from at least one of NeuroHIV Disease, Alzheimer's Disease, Parkinson Disease, Amyotrophic lateral sclerosis (ALS), multiple sclerosis (MS), or CNS disease with high circulating levels of ATP.

20. The method of claim 14, wherein the compound, peptide, protein, or nucleic acid expression vector is targeted to prevent at least one of: blood brain barrier (BBB) overactivation or CNS compromise.
